(19) United States
(12) Patent Application Publication
(10) Pub. No.: US 2024/0409662 A1
(43) Pub. Date: Dec. 12, 2024

(54) ANTIBODY MASKS AND USES THEREOF

(71) Applicant: Vibrant Pharma Limited

(72) Inventors: Jianbing Zhang, Guangzhou (CN); Hong Zhang, Guangzhou (CN); Luquan Wang, Guangzhou (CN)

(21) Appl. No.: 18/698,866

(22) PCT Filed: Dec. 9, 2022
(86) PCT No.: PCT/CN2022/137977

§ 371 Date: Apr. 5, 2024

(30) Foreign Application Priority Data
Dec. 9, 2021 (WO) PCT/CN2021/136652

(51) Int. Cl. C07K 16/30; C07K 14/725; C07K 16/28

(52) U.S. Cl. CPC C07K 16/3007; C07K 14/7051; C07K 16/2809; C07K 2317/31; C07K 2317/524; C07K 2317/526; C07K 2317/53; C07K 2317/55; C07K 2317/569; C07K 2317/622; C07K 2317/92; C07K 2319/32; C07K 2319/50

(57) ABSTRACT

Provided is a masking peptide for an antibody or antigen-binding fragment thereof. Also provided are polypeptides (e.g., antibody masks) that block the binding between an antibody (e.g., an anti-CD3 antibody) and its target, fusion proteins, and protein constructs and uses thereof.

### CROSS-REFERENCE TO RELATED APPLICATIONS

This application is a National Stage Application under 35 U.S.C. § 371 of International Patent Application No. PCT/CN2022/137977, filed on Dec. 9, 2022, which claims the benefit of priority to International Patent Application No. PCT/CN2021/136652, filed on Dec. 9, 2021, the contents of which are hereby incorporated by reference.

### SEQUENCE LISTING

This application contains a Sequence Listing that has been submitted electronically as an XML file named “51857-0007US1_SL_ST26.xml.” The XML file, created on Dec. 22, 2022, is 114,775 bytes in size. The material in the XML file is hereby incorporated by reference in its entirety.

### TECHNICAL FIELD

This disclosure relates to polypeptides (e.g., antibody masks) that block the binding between an antibody (e.g., an anti-CD3 antibody) and its target, fusion proteins, and protein constructs and uses thereof.

### BACKGROUND

Harnessing the power of immune cells, especially T cells, to enhance anti-tumor activities has become a promising strategy in clinical management of diseases and cancers such as hematologic malignancies.

A T-cell engager (TCE) is a protein that simultaneously binds through a target antigen on a tumor cell and a molecule (e.g., CD3) on a T-cell to form a TCR-independent artificial immune synapse and circumvent HLA restriction. The earliest efforts using CD3 binding antibodies for T-cell activation date back the mid-1980's when studies of heteroaggregates of anti-CD3 (T3, from OKT3 hybridoma) showed anti-cancer cytotoxicity (see, e.g., Perez Pet al., Specific lysis of human tumor cells by T cells coated with anti-T3 cross-linked to anti-tumor antibody. J Immunol October (1986) 137:2069-72). The first published description of a bispecific TCE was of a rat isotype hybrid generated by Clark and Waldmann (see, e.g., Clark M R et al., T-cell killing of target cells induced by hybrid antibodies: comparison of two bispecific monoclonal antibodies. J Natl Cancer Inst. (1987) 79:1393-401), who demonstrated targeted killing of TH-1 cells. After a lull in clinical development of bispecifics due in large part to manufacturing complications, the field witnessed the clinical success of a series bispecific T-cell engagers.

While these early studies showed promising clinical efficacy, they were also hampered by severe dose-limiting toxicities primarily manifesting as on-target-off-tumor toxicity (OTOT) as well as cytokine release syndrome (CRS). This resulted in prohibitively narrow therapeutic windows and was due in large part to the anti-CD3 binding domains that were used. Therefore, there exists a need for more effective therapies, especially with bispecific therapeutics good efficacy but lowered toxicity.

### SUMMARY

This disclosure provides polypeptides (e.g., antibody masks) that block the binding between an antibody (e.g., an anti-CD3 antibody) and its target, and fusion proteins, and protein constructs, and also the methods of inducing T cell immunity and treating cancer.

In one aspect, the disclosure relates to a protein construct comprising: a. a masking peptide comprising an amino acid sequence that is at least 80% identical to the sequence of SEQ ID NO: 3 or a portion thereof, wherein the masking peptide comprises one or more amino substitutions; and b.an antigen-binding domain, wherein the masking peptide and the antigen-binding domain are linked via a linker.

In some embodiments, the masking peptide comprises or consists of at least 3 amino acids (e.g., from N-terminal of CD3 [symbol] ).

In some embodiments, the masking peptide comprises or consists of any one of SEQ ID NOs: 60-68, and wherein the masking peptide comprises an amino substitution at a position corresponding to amino acid 1, 2, or 3 of SEQ ID NO: 3.

In some embodiments, the amino acid substitution is one of the following:
(1) a substitution of Q at the position corresponding to amino acid 1 of SEQ ID NO: 3 with C;
(2) a substitution of D at the position corresponding to amino acid 2 of SEQ ID NO: 3 with E, G, N, or C; and
(3) a substitution of G at the position corresponding to amino acid 3 of SEQ ID NO: 3 with A or R.

In some embodiments, the amino acid substitution is one of the following:
(1) a substitution of D at the position corresponding to amino acid 2 of SEQ ID NO: 3 with E, G, or N; and
(2) a substitution of G at the position corresponding to amino acid 3 of SEQ ID NO: 3 with A or R.

In some embodiments, the amino acid substitution is one of the following:
(1) a substitution of D at the position corresponding to amino acid 2 of SEQ ID NO: 3 with E, or G; and
(2) a substitution of G at the position corresponding to amino acid 3 of SEQ ID NO: 3 with A.

In some embodiments, the masking peptide comprises or consists of any one of SEQ ID NOs: 60-68 with at most one amino acid substitution at positions corresponding to amino acid 1-3 of SEQ ID NO: 3.

In some embodiments, the masking peptide comprises or consists of any one of SEQ ID NOs: 74, 76, 78, and 80-83.

In some embodiments, the linker comprises about 4 to about 18 amino acids. In some embodiments, the linker comprises a protease cleavable sequence. In some embodiments, the protease cleavable sequence comprises an amino acid sequence of PLGL (SEQ ID NO: 85).

In some embodiments, the linker comprises an amino acid sequence of X1-PLGL-X2, wherein X1 comprises 0-8 glycine (G), and/or 0-8 serine (S); X2 comprises 0-8 glycine (G), and/or 0-8 serine (S).

In some embodiments, the linker comprises or consists of any one of SEQ ID NOs: 84-91 (e.g., any one of SEQ ID NO: 86-91).

In some embodiments, the masking peptide and the linker together comprise an amino acid sequence that is at least 80% identical to any one of SEQ ID NOs: 24, 26-30, 32-36, and 38-45.

In some embodiments, the antigen-binding domain comprises a VH and a VL. In some embodiments, the masking peptide is linked to the VH (e.g., N-terminal of the VH) through the linker. In some embodiments, the masking peptide is linked to the VL (e.g., N-terminal of the VL) through the linker.

In some embodiments, the protein construct comprises two masking peptides, wherein one of the two masking peptides is linked to the VH through a first linker, and the other of the two masking peptides is linked to the VL through a second linker.

In some embodiments, the antigen-binding domain comprises a scFv. In some embodiments, the antigen-binding domain comprises a Fab. In some embodiments, the masking peptide is linked to the scFv (e.g., N-terminal of the scFv) through the linker.

In some embodiments, the antigen-binding domain comprises a VHH. In some embodiments, the masking peptide is linked to the VHH (e.g., N-terminal of VHH) through the linker.

In some embodiments, the antigen-binding domain specifically binds to CD3. In some embodiments, the antigen-binding domain specifically binds to one or more epitopes in any one of SEQ ID NOs: 60-68.

In some embodiments, the protein construct further comprises an antigen-binding domain that specifically binds to a tumor associated antigen. In some embodiments, the tumor-associated antigen is carcinoembryonic antigen cell adhesion molecule 5 (CEACAM5).

In one aspect, the disclosure relates to a protein construct comprising: (1) a first moiety comprising a masking peptide comprising an amino acid sequence that is at least 80% identical to the sequence of SEQ ID NO: 3 or a portion thereof, wherein the masking peptide comprises one or more amino substitutions; (2) a second moiety that specifically binds to CD3; and (3) a third moiety that specifically binds to a tumor associated antigen, wherein the first moiety and the second moiety are linked via a linker.

In some embodiments, the masking peptide comprises or consists of at least 3 amino acids (e.g., from N-terminal of CD3 [symbol] ).

In some embodiments, the masking peptide comprises or consists of any one of SEQ ID NOs: 60-68, and wherein the masking peptide comprises an amino substitution at a position corresponding to amino acid 1, 2, or 3 of SEQ ID NO: 3.

In some embodiments, the amino acid substitution is one of the following:
(1) a substitution of Q at the position corresponding to amino acid 1 of SEQ ID NO: 3 with C;
(2) a substitution of D at the position corresponding to amino acid 2 of SEQ ID NO: 3 with N, E, G, or C; and
(3) a substitution of G at the position corresponding to amino acid 3 of SEQ ID NO: 3 with A or R.

In some embodiments, the amino acid substitution is one of the following:
(1) a substitution of D at the position corresponding to amino acid 2 of SEQ ID NO: 3 with E, G, or N; and
(2) a substitution of G at the position corresponding to amino acid 3 of SEQ ID NO: 3 with A or R.

In some embodiments, the amino acid substitution is one of the following:
(1) a substitution of D at the position corresponding to amino acid 2 of SEQ ID NO: 3 with E, or G; and
(2) a substitution of G at the position corresponding to amino acid 3 of SEQ ID NO: 3 with A.

In some embodiments, the masking peptide comprises or consists of any one of SEQ ID NO: 74, 76, 78, and 80-83.

In some embodiments, the linker comprises a protease cleavable sequence. In some embodiments, the protease cleavable sequence comprises an amino acid sequence of PLGL (SEQ ID NO: 85). In some embodiments, the linker comprises an amino acid sequence of X1-PLGL-X2, wherein X1 comprises 0-8 glycine (G), and/or 0-8 serine (S); X2 comprises 0-8 glycine (G), and/or 0-8 serine (S). In some embodiments, the linker comprises or consists of any one of SEQ ID NOs: 84-91 (e.g., any one of SEQ ID NO: 86-91).

In some embodiments, the second moiety comprises an antibody or antigen-binding fragment thereof. In some embodiments, the second moiety comprises an antigen-binding fragment of an antibody. In some embodiments, the second moiety comprises a VHH. In some embodiments, the third moiety comprises an antibody or antigen-binding fragment thereof.

In some embodiments, the third moiety comprises an antigen-binding fragment of an antibody. In some embodiments, the third moiety comprises a VHH.

In some embodiments, the tumor associated antigen is selected from the group consisting of CD2, CD4, CD19, CD20, CD22, CD23, CD30, CD33, CD37, CD40, CD44v6, CD52, CD56, CD70, CD74, CD79a, CD80, CD98, CD138, EGFR (Epidermal growth factor receptor), VEGF (Vascular endothelial growth factor), VEGFR1 (Vascular endothelial growth factor receptor 1), PDGFR (Platelet-derived growth factor receptor), RANKL (Receptor activator of nuclear factor kappa-B ligand), GPNMB (Transmembrane glycoprotein Neuromedin B), EphA2 (Ephrin type-A receptor 2), PSMA (Prostate-specific membrane antigen), Cripto (Cryptic family protein 1B), EpCAM (Epithelial cell adhesion molecule), CTLA4 (Cytotoxic T-Lymphocyte Antigen 4), IGF-IR (Type 1 insulin-like growth factor receptor), GP3 (M13 bacteriophage), GP9 (Glycoprotein IX), CD42a, GP 40 (Glycoprotein 40 kDa), GPC3 (glypican-3), GPC1 (glypican-1), TRAILR1 (Tumor necrosis factor-related apoptosis-inducing ligand receptor 1), TRAILRII (Tumor necrosis factor-related apoptosis-inducing ligand receptor II), FAS (Type II transmembrane protein), PS (phosphatidyl serine) lipid, Muc (Mucin 1, PEM), Muc18, CD146, α5β1 integrin, α4β1 integrin, αv integrin (Vitronectin Receptor), Chondrolectin, CAIX (Carbonic anhydrase IX), GD2 gangloside, GD3 gangloside, GM1 gangloside, Lewis Y antigen, Mesothelin, HER2 (Human Epidermal Growth factor 2), HER3, HER4, FN14 (Fibroblast Growth Factor Inducible 14), CS1 (Cell surface glycoprotein, CD2 subset 1, CRACC, SLAMF7, CD319), 41BB CD137, SIP (Siah-1 Interacting Protein), CTGF (Connective tissue growth factor), HLADR (MHC class II cell surface receptor), PD-1 (Programmed Death 1, Type I membrane protein, PD-L1 (Programmed Death Ligand 1), PD-L2 (Programmed Death Ligand 2), IL-2 (Interleukin-2), IL-8 (Interleukin-8), IL-13 (Interleukin-13), PIGF (Phosphatidylinositol-glycan biosynthesis class F protein), NRP1 (Neuropilin-1), ICAM1, CD54, GC182 (Claudin 18.2), Claudin, HGF (Hepatocyte growth factor), CEA (Carcinoembryonic antigen), LTβR (lymphotoxin β receptor), Kappa Myeloma, Folate Receptor alpha, GRP78 (BIP, 78 kDa Glucose-regulated protein), A33 antigen, PSA (Prostate-specific antigen), CA 125 (Cancer antigen 125 or carbohydrate antigen 125), CA19.9, CA15.3, CA242, leptin, prolactin, osteopontin, IGF-II (Insulin-like growth factor 2), fascin, sPIgR (secreted chain of polymorphic immunoglobulin receptor), 14-3-3 eta protein, 5T4, ETA (epithelial tumor antigen), MAGE (Melanoma-associated antigen), MAPG (Melanoma-associated proteoglycan, NG2), vimentin, EPCA-1 (Early prostate cancer antigen-2), TAG-72 (Tumor-associated glycoprotein 72), factor VIII, Neprilysin (Membrane metallo-endopeptidase), 17-1 A (Epithelial cell surface antigen 17-1A), nucleolin, nucleophosmin, and any combination thereof.

In some embodiments, the tumor associated antigen is carcinoembryonic antigen cell adhesion molecule 5 (CEACAM5). In some embodiments, the second moiety is linked to a first CH2 domain and a first CH3 domain.

In some embodiments, the second moiety is linked to the first CH2 domain and the first CH3 domain through a IgG hinge region. In some embodiments, the third moiety is linked to a second CH2 domain and a second CH3 domain.

In some embodiments, the third moiety is linked to the second CH2 domain and the second CH3 domain through a IgG hinge region. In some embodiments, the first and the second CH3 domains have one or more knobs-into-holes mutations.

In one aspect, the disclosure relates to a polynucleotide encoding any of the protein constructs disclosed herein.

In one aspect, the disclosure relates to a vector comprising the polynucleotide disclosed herein.

In one aspect, the disclosure relates to a cell comprising the vector disclosed herein.

In one aspect, the disclosure relates to a method of producing a protein construct, the method comprising (a) culturing the cell disclosed herein under conditions sufficient for the cell to produce the protein construct; and (b) collecting the protein construct produced by the cell.

In one aspect, the disclosure relates to a method of inducing or activating a T cell immunity in a subject, comprising administering an effective amount of any of the protein constructs disclosed herein to the subject.

In some embodiments, the T cell immunity is induced or activated upon the cleavage of the protease cleavable domain of the linker.

In some embodiments, the linker is cleaved at a site of abundant protease activity.

In some embodiments, the site of abundant protease activity is a tumor microenvironment. In some embodiments, the protease is MMP9.

In one aspect, the disclosure relates to a method of inhibiting tumor growth in a subject, comprising administering an effective amount of any of the protein constructs disclosed herein to the subject.

In one aspect, the disclosure relates to a method of treating a cancer in a subject, comprising administering an effective amount of any of the protein constructs disclosed herein to the subject.

In some embodiments, the method described herein further comprises administering an additional therapeutic agent to the subject.

In one aspect, the disclosure relates to a masking peptide comprising:
X₁X₂X₃NE (SEQ ID NO: 92) or X₁X₂X₃NEE (SEQ ID NO: 93), wherein X₁ is Q, or C; X₂ is D, E, G, N, or C; and X₃ is G, A or R. In some embodiments, X₁ is Q; X₂ is D, E, G, or N; and X₃ is G, A or R. In some embodiments, X₁ is Q; X₂ is D, E, or G; and X₃ is G, or A. In some embodiments, X₁X₂X₃NE (SEQ ID NO: 92) or X₁X₂X₃NEE (SEQ ID NO: 93) in the masking peptide is not identical to QDGNE (SEQ ID NO: 60) or QDGNEE (SEQ ID NO: 68). In some embodiments, X₁X₂X₃ in the masking peptide differs from QDG by one amino acid.

In one aspect, the disclosure relates to a protein construct comprising: (a) a masking peptide comprising an amino acid sequence that is at least 80% identical to the sequence of SEQ ID NO: 3 or a portion thereof, and (b) an antigen-binding domain, wherein the masking peptide and the antigen-binding domain are linked via a linker.

In some embodiments, the masking peptide comprises or consists of at least 3, 4, 5, 6, 7, 8, 9, or 10 amino acids (e.g., from N-terminal of CD3e).

In some embodiments, the masking peptide comprises or consists of any one of SEQ ID NOs: 60-68.

In some embodiments, the masking peptide comprises or consists of any one of SEQ ID NOs: 60-68 with one or more amino substitutions.

In some embodiments, the one or more amino acid substitutions comprise one or more of the following: (1) a substitution of Q at the position corresponding to amino acid 1 of SEQ ID NO: 3 with E, S, Y, H, or V; (2) a substitution of D at the position corresponding to amino acid 2 of SEQ ID NO: 3 with N, R, E, I, or G; and (3) a substitution of G at the position corresponding to amino acid 3 of SEQ ID NO: 3 with I, A or R.

In some embodiments, the one or more amino acid substitutions comprise one or more of the following: (1) a substitution of Q at the position corresponding to amino acid 1 of SEQ ID NO: 3 with C; (2) a substitution of D at the position corresponding to amino acid 2 of SEQ ID NO: 3 with E, G, N, or C; and (3) a substitution of G at the position corresponding to amino acid 3 of SEQ ID NO: 3 with A or R.

In some embodiments, the one or more amino acid substitutions comprise one or more of the following: (1) a substitution of D at the position corresponding to amino acid 2 of SEQ ID NO: 3 with E, G, or N; and (2) a substitution of G at the position corresponding to amino acid 3 of SEQ ID NO: 3 with A or R.

In some embodiments, the one or more amino acid substitutions comprise one or more of the following: (1) a substitution of D at the position corresponding to amino acid 2 of SEQ ID NO: 3 with E, or G; and (2) a substitution of G at the position corresponding to amino acid 3 of SEQ ID NO: 3 with A.

In some embodiments, the masking peptide comprises or consists of any one of SEQ ID NOs: 60-68 with at most one amino acid substitution or at most two amino acid substitutions.

In some embodiments, the masking peptide comprises or consists of any one of SEQ ID NO: 69-83.

In some embodiments, the linker comprises about 8 to about 18 amino acids.

In some embodiments, the linker comprises a protease cleavable sequence.

In some embodiments, the protease cleavable sequence comprises an amino acid sequence of PLGL (SEQ ID NO: 85).

In some embodiments, the linker comprises an amino acid sequence of X1-PLGL-X2, wherein X1 comprises 0-8 glycine (G), and/or 0-8 serine (S); X2 comprises 0-8 glycine (G), and/or 0-8 serine (S).

In some embodiments, the linker comprises or consists of any one of SEQ ID NOs: 84-91 (e.g., any one of SEQ ID NO: 86-91).

In some embodiments, the masking peptide and the linker together comprise an amino acid sequence that is at least 80% identical to any one of SEQ ID NOs: 12-45.

In some embodiments, the antigen-binding domain comprises a VH and a VL.

In some embodiments, the masking peptide is linked to the VH (e.g., N-terminal of the VH) through the linker.

In some embodiments, the masking peptide is linked to the VL (e.g., N-terminal of the VL) through the linker.

In some embodiments, the protein construct comprises two masking peptides, wherein one of the two masking peptides is linked to the VH through a first linker, and the other of the two masking peptides is linked to the VL through a second linker.

In some embodiments, the antigen-binding domain comprises a scFv.

In some embodiments, the masking peptide is linked to the scFv (e.g., N-terminal of the scFv) through the linker.

In some embodiments, the antigen-binding domain comprises a VHH.

In some embodiments, the masking peptide is linked to the VHH (e.g., N-terminal of VHH) through the linker.

In some embodiments, the antigen-binding domain specifically binds to CD3.

In some embodiments, the protein construct further comprises an antigen-binding domain that specifically binds to a tumor associated antigen.

In some embodiments, the tumor-associated antigen is carcinoembryonic antigen cell adhesion molecule 5 (CEACAM5).

In one aspect, the disclosure relates to a protein construct comprising: (1) a first moiety comprising a masking peptide comprising an amino acid sequence that is at least 80% identical to the sequence of SEQ ID NO: 3 or a portion thereof, (2) a second moiety that specifically binds to CD3; and (3) a third moiety that specifically binds to a tumor associated antigen, wherein the first moiety and the second moiety are linked via a linker.

In some embodiments, the masking peptide comprises or consists of at least 5 amino acids (e.g., from N-terminal of CD3 [symbol] ).

In some embodiments, the masking peptide comprises or consists of any one of SEQ ID NOs: 60-68, optionally with one or more amino substitutions.

In some embodiments, the one or more amino acid substitutions comprise one or more of the following: (1) a substitution of Q at the position corresponding to amino acid 1 of SEQ ID NO: 3 with E, S, Y, H, or V; (2) a substitution of D at the position corresponding to amino acid 2 of SEQ ID NO: 3 with N, R, E, I, or G; and (3) a substitution of G at the position corresponding to amino acid 3 of SEQ ID NO: 3 with I, A or R.

In some embodiments, the one or more amino acid substitutions comprise one or more of the following: (1) a substitution of D at the position corresponding to amino acid 2 of SEQ ID NO: 3 with E, or G; and (2) a substitution of G at the position corresponding to amino acid 3 of SEQ ID NO: 3 with A.

In some embodiments, the masking peptide comprises or consists of any one of SEQ ID NO: 69-83.

In some embodiments, the linker comprises a protease cleavable sequence.

In some embodiments, the protease cleavable sequence comprises an amino acid sequence of PLGL (SEQ ID NO: 85).

In some embodiments, the linker comprises an amino acid sequence of X1-PLGL-X2, wherein X1 comprises 0-8 glycine (G), and/or 0-8 serine (S); X2 comprises 0-8 glycine (G), and/or 0-8 serine (S).

In some embodiments, the linker comprises or consists of any one of SEQ ID NOs: 84-91 (e.g., any one of SEQ ID NO: 86-91).

In some embodiments, the second moiety is an antibody or antigen-binding fragment thereof. In some embodiments, the second moiety comprises an antigen-binding fragment (Fab) of an antibody. In some embodiments, the second moiety comprises a VHH.

In some embodiments, the third moiety is an antibody or antigen-binding fragment thereof. In some embodiments, the third moiety comprises an antigen-binding fragment (Fab) of an antibody. In some embodiments, the third moiety comprises a VHH.

In some embodiments, the tumor associated antigen is carcinoembryonic antigen cell adhesion molecule 5 (CEACAM5).

In some embodiments, the second moiety is linked to a first CH2 domain and a first CH3 domain. In some embodiments, the second moiety is linked to the first CH2 domain and the first CH3 domain through a IgG hinge region.

In some embodiments, the third moiety is linked to a second CH2 domain and a second CH3 domain. In some embodiments, the third moiety is linked to the second CH2 domain and the second CH3 domain through a IgG hinge region.

In some embodiments, the first and the second CH3 domains have one or more knobs-into-holes mutations.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (V_{H}H) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain, or a VHH). Non-limiting examples of antibody fragments include, e.g., Fab, Fab′, F(ab′)2, and Fv fragments, scFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to human CD3 may be referred to as a CD3-specific antibody or an anti-CD3 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain only antibody. In some embodiments, the VHH is a humanized VHH. In some embodiments, the VHH is a single-domain antibody (sdAb).

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A shows the BLI sensorgrams of binding of the anti-CD3 antibody SP34 with CD3δε at concentrations of 9.4, 18.8, 37.5, 75.0 and 150 nM, respectively.

FIG. 1B shows the sequences of the full length CD3ε (SEQ ID NO: 3) and eight peptides of a portion of human CD3 [symbol] , ranging from 5-aa to 27-aa long with their C-terminus linked to a GGGGS linker (SEQ ID NOs: 4-11) and biotinylated.

FIG. 1C shows the bio-layer interferometry (BLI) sensorgrams of binding of CD3ε peptides to SP34.

FIG. 2A shows the binding of SP34 to wild type and 15 mutants of CD3 [symbol] 1-27-Fc fusion proteins. Mutants (shown in the figure) were coated as the antigen, and SP34 was used as the detection antibody in an ELISA assay.

FIG. 2B shows the binding of sensorgrams of six out of 42 CD3 [symbol] 1-27-Fc fusion proteins to SP34.

FIG. 3A is a set of schematics of masked TCE (M-TCE) provided herein. Three typical M-TCEs (left, mask on the heavy chain; middle, mask on the light chain; right, masks on both chains) are shown. SP34 Fab (SP34) together with its mask (Mask) and a protease digestible linker (PDL) is constructed on the knob chain (K), and an sdAb binding to CEACAM5 (CEA) on the hole chain (H) of a knob-in-hole construct of human IgG (e.g., IgG4).

FIG. 3B shows the SDS-PAGE of 19 M-TCEs under reducing condition.

FIG. 3C shows the sensorgrams of the binding of six M-TCEs with wild type mask QDGNEE to CD36. Unmasked TCE SP34-CEA (upper left) was used as a control.

FIG. 4 shows the binding sensorgrams of 14 M-TCEs to SP34.

FIG. 5A shows the SDS-PAGE of seven M-TCEs before (B) and after (A) MMP9 digestion.

FIG. 5B shows the binding sensorgrams of the seven M-TCEs. Top panel, non-digested M-TCE binding to the antigen CEACAM5, middle panel, non-digested M-TCE binding to CD36; bottom panel, digested M-TCE binding to CD336.

FIGS. 6A-6E show the efficacy of five M-TCEs before and after the removal of the mask. The efficacy is shown by M-TCE-induced killing of HT29 cells by L4-G3A, L4-D2E, L5-D2E, L6-D2E and L7-D2G in the presence of human PBMC. Non-masked TCE TA1F-CEA was used as a control.

FIG. 7 shows the characterization of three types of M-TCEs with a mask fused to SP34 on its heavy chain, light chain or both chains. Top panels are representative sensorgrams of bindings between the three M-TCEs together with a non-masked control TCE SP34-CEA to CD3δε before MM9 digestion; Lower panel are representative sensorgrams of bindings between the three M-TCEs together with a non-masked control TCE SP34-CEA to CD3δε after MM9 digestion.

FIG. 8 lists sequences in the disclosure.

### DETAILED DESCRIPTION

CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex.

This disclosure provides polypeptides (e.g., antibody masks) that block the binding between an antibody (e.g., an anti-CD3 antibody) and its target, fusion proteins, and protein constructs that bind to human CD3. These fusion proteins and protein constructs can be used to induce T cell immunity and treat cancer.

### Antibody Masking Peptides

The present disclosure provides antibody masks that block the binding of an antibody to its target. In some embodiments, the antibody is an anti-CD3 antibody. In some embodiments, the antibody masks are polypeptides. In some embodiments, the antibody masking peptide includes at least a portion of the epitope of the antibody. For example, a masking peptide provided herein can be a polypeptide including an amino acid sequence that is at least 50%, 55%, 60%, 65%, 70%, 75%, 80%, 85%, 90%, 95%, or 100% identical to the epitope of an anti-CD3 antibody. In some embodiments, the epitope is human CD3F or a portion thereof. The amino acid sequence of human CD3F is shown in SEQ ID NO:3.

QDGNEEMGGITQTPYKVSISGTTVILTCPQYPGSEILWQHNDKNIGGDEDDKN IGSDEDHLSLKEFSELEQSGYYVCYPRGSKPEDANFYLYLRARV (SEQ ID NO: 3)

In some embodiments, the epitope of the anti-CD3 antibody is any portion (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 97 amino acids) of the sequence of SEQ ID NO: 3.

In some embodiments, the masking peptide described herein is a polypeptide that is about or at least 1, 2, 3, 4, 5, 6, 7, 8, 9, 10, 11, 12, 13, 14, 15, 16, 17, 18, 19, 20, 21, 22, 23, 24, 25, 26, 27, 30, 35, 40, 45, 50, 55, 60, 65, 70, 75, 80, 85, 90, 95, or 97 amino acids long. In some embodiments, the masking peptide is about 4 to about 30 amino acids long. In some embodiments, the masking peptide is about 5 to about 27 amino acids long. In some embodiments, the masking peptide has about 5 to about 20 amino acids, about 5 to about 15 amino acids, about 5 to about 10 amino acids, about 5 to about 7 amino acids, or about 5 to about 6 amino acids.

In some embodiments, the masking peptide is a polypeptide that is at least 50%, 55%, 60%, 65%, 70%, 75%, 80%, 85%, 90%, 95%, or 100% identical to a portion (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 97 amino acids) of the sequence of SEQ ID NO: 3.

In some embodiments, the masking peptide comprises or consist of an amino acid sequence that is at least 80%, 85%, 90%, 95%, or 100% identical to residues 1-5 of SEQ ID NO:3. In some embodiments, the masking peptide has an amino acid sequence that is at least 80%, 85%, 90%, 95%, or 100% identical to residues 1-8 of SEQ ID NO:3. In some embodiments, the masking peptide has an amino acid sequence that is at least 80%, 85%, 90%, 95%, or 100% identical to residues 1-11 of SEQ ID NO:3. In some embodiments, the masking peptide has an amino acid sequence that is at least 80%, 85%, 90%, 95%, or 100% identical to residues 1-14 of SEQ ID NO:3. In some embodiments, the masking peptide has an amino acid sequence that is at least 80%, 85%, 90%, 95%, or 100% identical to residues 1-17 of SEQ ID NO:3. In some embodiments, the masking peptide has an amino acid sequence that is at least 80%, 85%, 90%, 95%, or 100% identical to residues 1-20 of SEQ ID NO:3. In some embodiments, the masking peptide has an amino acid sequence that is at least 80%, 85%, 90%, 95%, or 100% identical to residues 1-23 of SEQ ID NO:3. In some embodiments, the masking peptide has an amino acid sequence that is at least 80%, 85%, 90%, 95%, or 100% identical to residues 1-27 of SEQ ID NO:3.

In some embodiments, the masking peptide described herein has one or more amino acid substitution compared to the sequence (or the portion thereof) of SEQ ID NO: 3. In some embodiments, the one or more amino acid substitution is in amino acid residue 1, 2, and/or 3 of SEQ ID NO: 3. In some embodiments, the one or more amino acid substitution is selected from the group consisting Q1E, Q1S, Q1Y, Q1H, Q1V, Q1C, D2N, D2R, D2E, D2I, D2G, D2C, G3I, G3A, and G3R. In some embodiments, the masking peptide includes 1, 2, 3, 4, 5, or more amino acid substitutions compared to the sequence (or the portion thereof) of SEQ ID NO: 3.

In some embodiments: the amino acid residue corresponding to position 1 of SEQ ID NO: 3 is Q, E, S, Y, H, or V; the amino acid residue corresponding to position 2 of SEQ ID NO:3 is D, N, R, E, I, or G; and the amino acid residue corresponding to position 3 of SEQ ID NO:3 is G, A or R.

In some embodiments, the masking peptide comprises one or more amino acid substitutions selected from the following: Q1C, D2E, D2G, D2N, D2C, G3A, or G3R.

In some embodiments, the masking peptide comprises one or more amino acid substitutions selected from the following: D2E, D2G, D2N, G3A, or G3R.

In some embodiments, the masking peptide comprises one or more amino acid substitutions selected from the following: D2E, D2G, or G3A.

In some embodiments, the masking peptide comprises or consist of: X₁X₂X₃NE (SEQ ID NO: 92) or X₁X₂X₃NEE (SEQ ID NO: 93), wherein X₁ is Q, or C; X₂ is D, E, G, N, or C; and X₃ is G, A or R. In some embodiments, X₁ is Q; X₂ is D, E, G, or N and X₃ is G, A or R. In some embodiments, X₁ is Q; X₂ is D, or E; and X₃ is G, A or R. In some embodiments, X₁ is Q; X₂ is D, G or E; and X₃ is G or A. In some embodiments, the masking peptide does not comprise QDGNE (SEQ ID NO: 60) or QDGNEE (SEQ ID NO: 68).

Also provided herein are fusion peptides including the masking peptide described herein fused to an Fc region of an immunoglobulin. In some embodiments, the Fc region is a human Fc region. In some embodiments, the masking peptide is fused to the human Fc region via a linker. Any suitable linker can be used to fuse the masking peptide and the Fc region. In some embodiments, the human Fc region further includes a hinge region. In some embodiments, the hinge region is a human IgG4 hinge region.

Also provided herein are polypeptides including (1) an masking peptide described herein and (2) a linker.

In some embodiments, the linker is a cleavable linker. In some embodiments, the linker includes a protease cleavable sequence. Any suitable protease cleavable sequence can be used herein. In some embodiments, the protease cleavable sequence includes an amino acid sequence of PLGL (SEQ ID NO: 85). In some embodiments, the protease cleavable sequence is cleavable by a protease (e.g., Matrix metalloproteinase 9 (MMP9) protease).

In addition to the protease cleavable sequence, the linker used herein can further include additional amino acid sequences. In some embodiments, the linker has an amino acid sequence of X1-PLGL-X2, wherein X1 comprises 0-8 glycine (G), and/or 0-8 serine (S); and X2 comprises 0-8 glycine (G), and/or 0-8 serine (S).

In some embodiments, the cleavable linker has a sequence of PLGL (SEQ ID NO: 85). In some embodiments, the cleavable linker has a sequence of GSPLGLGS (SEQ ID NO: 86). In some embodiments, the cleavable linker has a sequence of SGSPLGLSGG (SEQ ID NO: 87). In some embodiments, the cleavable linker has a sequence of GSGSPLGLGSGS (SEQ ID NO: 88). In some embodiments, the cleavable linker has a sequence of SSGGSPLGLSSGGS (SEQ ID NO: 89). In some embodiments, the cleavable linker has a sequence of SSGGGSPLGLSSGGGS (SEQ ID NO: 90). In some embodiments, the cleavable linker has a sequence of SSGGSGSPLGLSSGGSGS (SEQ ID NO: 91).

In some embodiments, the linker has a sequence of GGGGS (SEQ ID NO: 84) or multiple copies of GGGGS (SEQ ID NO: 84).

The linker described herein can be any suitable length. In some embodiments, the linker is about 1, 2, 3, 4, 5, 6, 7, 8, 9, 10, 11, 12, 13, 14, 15, 16, 17, 18, 19, 20 or more amino acids long. In some embodiments, the linker has about 8 to about 18 amino acids, about 8 to about 15 amino acids, about 8 to about 10 amino acids, about 10 to about 18 amino acids, or about 10 to about 15 amino acids. In some embodiments, the linker has about 4 to about 18 amino acids, about 4 to about 15 amino acids, or about 4 to about 10 amino acids.

In some embodiments, the polypeptide described herein has an amino acid sequence that is at least 50%, 55%, 60%, 65%, 70%, 75%, 80%, 85%, 90%, 95%, or 100% identical to any one of SEQ ID NOs: 4-45. In some embodiments, the polypeptide described herein has an amino acid sequence that is at least 50%, 55%, 60%, 65%, 70%, 75%, 80%, 85%, 90%, 95%, or 100% identical to any one of SEQ ID NOs: 4-11. In some embodiments, the polypeptide described herein has an amino acid sequence that is at least 50%, 55%, 60%, 65%, 70%, 75%, 80%, 85%, 90%, 95%, or 100% identical to any one of SEQ ID NOs: 12-45.

In some embodiments, the polypeptide described herein is about 3, 4, 5, 6, 7, 8, 9, 10, 11, 12, 13, 14, 15, 15, 17, 18, 19, 20, 25, 30, 35, 40, 45, 50, 55, 60 or more amino acids long. In some embodiments, the polypeptide described herein is about 3 to about 30 amino acids long. In some embodiments, the polypeptide described herein is about 4 to about 30 amino acids long. In some embodiments, the polypeptide described herein is about 3 to about 97 amino acids long. In some embodiments, the polypeptide described herein has less than 97 amino acids.

In some embodiments, the masking peptide comprises or consists of the amino acid sequence of at least amino acid residues 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, or 1-30 of SEQ ID NO: 3. In some embodiments, the amino acid sequence corresponding to amino acid residues 1-3 of SEQ ID NO: 3 comprises exactly 1, 2, 3, 4, or 5 substitutions as described herein. In some embodiments, the amino acid sequence has exactly 1 or 2 substitutions (e.g., 1 substitution) at the first 3 amino acids of the N-terminal of SEQ ID NO: 3.

In some embodiments, the masking peptide comprises the amino acid sequence of at least amino acid residues 1-3 of SEQ ID NO: 3. In some embodiments, the amino acid sequence corresponding to amino acid residues 1-3 of SEQ ID NO: 3 comprises exactly 1 substitution as described herein.

In some embodiments, the masking peptide comprises or consists of any one of SEQ ID NOs: 60-68, with exactly 1 or 2 substitutions (e.g., 1 substitution) at the first 3 amino acids of the N-terminal of SEQ ID NOs: 60-68.

### Modified Antibodies or Antigen Binding Fragments Thereof

In one aspect, the masking peptide can be used for any antibody or antigen-binding fragment thereof as described herein. In the some embodiments, the masking peptide is linked to the antibody or antigen-binding fragment thereof via any one of the linkers as described herein. In some embodiments, the masking peptide is linked to an antigen binding domain, e.g., one or two masking peptides are linked to each antigen binding domain (e.g., that targets CD3) in the antibody.

Also provided herein are fusion proteins including (1) a first moiety comprising an masking peptide described herein; and (2) a second moiety comprising an antibody or antigen-binding fragment thereof that binds to human CD3, wherein the first moiety and the second moiety are linked via a linker.

In some embodiments, provided herein are fusion proteins comprising: (1) a first moiety comprising a polypeptide having an amino acid sequence that is at least 80% identical to the sequence of SEQ ID NO: 3 or a portion thereof, and (2) a second moiety comprising an antibody or antigen-binding fragment thereof that binds to human CD3, wherein the first moiety and the second moiety are linked via a linker.

An antibody can have two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, Antibody engineering, Springer Berlin Heidelberg, 2001. 422-439; Abhinandan, et al., Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. In some embodiments, the Kabat definition is used. In some embodiments, the Chothia definition is used.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In one aspect, the disclosure provides an antibody or an antigen binding fragment with one or more masking peptides, e.g., 1, 2, 3, 4 or more than 4 masking peptides as described herein. These masking peptides can be linked to the N-terminal of VH, and VL, or the C-terminal of VH and VL.

In some embodiments, one masking peptide is sufficient for an antigen binding domain. In some embodiments, a masking peptide is linked to the N-terminal of VH. In some embodiments, a masking peptide is linked to the N-terminal of VL.

In some embodiments, two masking peptides are used for one antigen binding domain. One is linked to the N-terminal of VH. The other is linked to the N-terminal of VL.

In one aspect, the protein construct comprises a masking peptide comprising the amino acid sequence of at least amino acid residues 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, or 1-30 of SEQ ID NO: 3, wherein the masking peptide comprises one or more amino substitutions; and an antigen-binding domain, wherein the masking peptide and the antigen-binding domain are linked via a linker. In some embodiments, the amino acid sequence corresponding to amino acid residues 1-3 of SEQ ID NO: 3 comprises exactly one substitution as described herein.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, Frontiers in immunology 5 (2014); Irani, et al., Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, a VHH, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, an individual CDR from either the heavy chain or the light chain of an intact antibody, or a VHH. In some embodiments, the antigen binding domain comprises a VH and a VL. In some embodiments, the antigen binding domain comprises a VHH.

In some embodiments, the antibody or antigen-binding fragments of the moiety in the protein construct described herein specifically binds to CD3 (e.g., human CD3). In some embodiments, the anti-CD3 antibody is SP34 (see, e.g., Pessano et al. The EMBO Journal. 4:337-344, 1985) or an antibody or antigen binding fragment derived from SP34. The amino acid sequence of the VH of SP34 is set forth in SEQ ID NO: 1. In some embodiments, the amino acid sequence of the VL of Sp34 is set forth in SEQ ID NO: 2. In some embodiments, the amino acid sequences of the VH CDR1, CDR2, and CDR3 are set forth in SEQ ID NOs: 46, 47 and 48, respectively. In some embodiments, the amino acid sequences of the VL CDR1, CDR2, and CDR3 are set forth in SEQ ID NOs: 49, 50 and 51, respectively.

Furthermore, in some embodiments, the antibodies or antigen-binding fragments thereof described herein can also contain one, two, or three heavy chain variable region CDRs selected from VH CDRs of SP34, and one, two, or three light chain variable region CDRs selected from VL CDRs of SP34. These CDR sequences can be determined by Kabat or Chothia definitions.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, 95%, 96%, 97%, 98%, 99% or 100% identical to the VH CDR1 amino acid sequence of SP34, the CDR2 region comprises or consists of an amino acid sequence that is at least 80%, 85%, 90%, 95%, 96%, 97%, 98%, 99% or 100% identical to the VH CDR2 amino acid sequence of SP34, and the CDR3 region comprises or consists of an amino acid sequence that is at least 80%, 85%, 90%, 95%, 96%, 97%, 98%, 99% or 100% identical to the VH CDR3 amino acid sequence of SP34. In some embodiments, the antibodies can have a light chain variable region (VL) comprising CDRs 1, 2, 3, wherein the CDR1 region comprises or consists of an amino acid sequence that is at least 80%, 85%, 90%, 95%, 96%, 97%, 98%, 99% or 100% identical to the VL CDR1 amino acid sequence of SP34, the CDR2 region comprises or consists of an amino acid sequence that is at least 80%, 85%, 90%, 95%, 96%, 97%, 98%, 99% or 100% identical to the VL CDR2 amino acid sequence of SP34, and the CDR3 region comprises or consists of an amino acid sequence that is at least 80%, 85%, 90%, 95%, 96%, 97%, 98%, 99% or 100% identical to the VL CDR3 amino acid sequence of SP34.

In some embodiments, the antibodies or antigen-binding fragments thereof contain a heavy chain variable region (VH) comprising or consisting of an amino acid sequence that is at least 80%, 85%, 90%, 95%, 96%, 97%, 98% or 99% identical to the VH sequence of SP34, and a light chain variable region (VL) comprising or consisting of an amino acid sequence that is at least 80%, 85%, 90%, 95%, 96%, 97%, 98% or 99% identical to the VL sequence of SP34.

In some embodiments, the antibody or antigen-binding fragment thereof of the moiety (e.g., targeting CD3) is a single-chain variable fragment (scFv), a single-domain antibody (sdAb), or a antigen-binding fragment (Fab).

In some embodiments, the antibodies or antigen-binding fragments thereof is a human antibody. In some embodiments, the antibodies or antigen-binding fragments thereof is a humanized antibody. Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al, MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the antibodies or antigen-binding fragments thereof cross-competes with SP34. In some embodiments, the antibodies or antigen-binding fragments thereof binds to the same epitope as SP34.

In some embodiments, the masking peptide described herein specifically binds to the linked antibody or antigen-binding fragment as described herein. In some embodiments, the masking peptide blocks the binding of the antibody or antigen-binding fragment to its target (e.g., a human CD3). The blocking can be measured by any suitable methods known in the art. In some embodiments, the blocking of binding is shown as reduced binding or affinity of the antibody or antigen-binding fragment to its target. In some embodiments, the binding or affinity is reduced by about 5%, 10%, 15%, 20%, 25%, 30%, 25%, 40%, 45%, 50%, 55%, 60%, 65%, 70%, 75%, 80%, 85%, 90%, 95% or more.

In some embodiments, the blocking of the binding of antibody or antigen-binding fragment to its target is reversible. In some embodiments, the blocking of the binding of antibody or antigen-binding fragment to its target is reversed by the cleavage of the linker as described herein.

In some embodiments, the masking peptide as described herein can be used in connection with the antigen binding domain in a chimeric antigen receptor. The chimeric antigen receptor or “CAR” refers to a fusion protein comprising an extracellular domain capable of binding to an antigen, and an intracellular region comprising one or more intracellular signaling domains derived from signal transducing proteins. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell.

### Protein Constructs

Also disclosed herein are protein constructs (e.g., bi-specific T-cell engagers), including: (1) a first moiety comprising an masking peptide described herein; (2) a second moiety comprising an antibody or antigen-binding fragment thereof that binds to human CD3, wherein the first moiety and the second moiety is linked via a linker; and (3) a third moiety that specifically binds to a tumor associated antigen (e.g., a tumor antigen). Also provided herein are protein constructs (e.g., bi-specific T-cell engagers), including (1) a first moiety comprising an amino acid sequence that is at least 80% identical to the sequence of SEQ ID NO: 3 or a portion thereof, (2) a second moiety comprising an antibody or antigen-binding fragment thereof that binds to human CD3, wherein the first moiety and the second moiety is linked via a linker; and (3) a third moiety that specifically binds to a tumor antigen.

Any antibody mask, antibody or antigen-binding fragment thereof can be used in the protein constructs described herein. Human crystallizable fragments (Fc) suitable for use in the protein constructs described herein are known in the art.

In some embodiments, the first and second moieties of the protein constructs (e.g., bi-specific T-cell engagers) disclosed herein are connected via a linker. Any suitable linkers known in the art and described herein can be used to connect the first and second moieties of the protein constructs described herein. In some embodiments, the link is a protease cleavable linker. In some embodiments, the linker is cleavable by an MMP9 protease.

In some embodiments, the protein constructs (e.g., bi-specific T-cell engagers) described herein has an amino acid sequence that is at least 80%, 85%, 90%, 95%, 96%, 97%, 98%, or 99% identical to the amino acid sequence of any one of the sequences as described herein.

In some embodiments, the antibody (antigen-binding fragments thereof, or molecules derived therefrom) specifically binds to the target (e.g., CD3) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms. Binding affinities can be deduced from the quotient of the kinetic rate constants (KD=koff/kon). In some embodiments, KD for the antibody, the antigen-binding fragments thereof, or molecules derived therefrom (e.g., CAR), is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. However, when the antigen binding domain is masked by a masking peptide, the binding affinity is reduced significantly. In some embodiments, the ratio of KD for a masked antigen binding domain and a unmasked (e.g., after being digested) antigen binding domain is at least 10, 50, 100, 200, 500, 1000, or 10000. In some embodiments, the masked antigen binding domain cannot bind to the antigen of interest (e.g., CD3).

General techniques for measuring the affinity of an antibody for an antigen include, e.g., ELISA, RIA, bio-layer interferometry (BLIand surface plasmon resonance (SPR).

In some embodiments, thermal stabilities are determined. The protein constructs as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, after cleavage, the protein constructs described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds, as compared to that of immune cells in the present of the masked protein constructs.

The tumor microenvironment usually has abundant protease activities. The protease can cleave the linker, and expose the antigen binding domain. In some embodiments, the protease cleavable domain of the linker is cleaved at a site of abundant endogenous protease. In some embodiments, the site of abundant endogenous protease is a tumor microenvironment. Proteases that are abundant at tumor microenvironment are known the art. The tumor microenvironment (TME) is a complex structure composed of a large variety of cell types embedded in a modified extracellular matrix (ECM), with bidirectional communication between cells and ECM macromolecules to determine tumor progression and metastatic dissemination. The communication may involve cell-cell contacts but may also be controlled by intact ECM macromolecules or by several of their domains released by limited proteolysis and called matrikines or matricryptins. ECM alterations occur in TME. In some embodiments, the protease used in the methods described herein is a protease that alters the ECM in tumor microenvironment (see, e.g., Brassart-Pasco et al., Front. Oncol., 15 Apr. 2020). Examples of such protease include, but are not limited to, cross-linking enzymes of the lysyl oxidase (LOX), transglutaminase families, particularly LOX-1, LOXL-2, and transglutaminase-2, matrix metalloprotease (MMP)-2, MMP-9, legumain asparaginyl endopeptidase, thrombin, fibroblast activation protease (FAP), MMP-1, MMP-3, MMP-7, MMP-8, MMP-12, MMP-13, MMP-14, membrane type 1 matrix metalloprotease (MT1-MMP), plasmin, transmembrane protease, serine (TMPRSS-3/4), cathepsin A, cathepsin B, cathepsin D, cathepsin E, cathepsin F, cathepsin H, cathepsin K, cathepsin L, cathepsin L2, cathepsin O, cathepsin S, caspase 1, caspase 2, caspase 3, caspase 4, caspase 5, caspase 6, caspase 7, caspase 8, caspase 9, caspase 10, caspase 11, caspase 12, caspase 13, caspase 14, human neutrophil elastase, urokinase/urokinase-type plasminogen activator (uPA), a disintegrin and metalloprotease (ADAM)10, ADAM12, ADAM17, ADAM with thrombospondin motifs (ADAMTS), ADAMTS5, beta secretase (BACE), granzyme A, granzyme B, guanidinobenzoatase, hepsin, matriptase, matriptase 2, meprin, neprilysin, prostate-specific membrane antigen (PSMA), tumor necrosis factor-converting enzyme (TACE), kallikrein-related peptidase (KLK)3, KLK5, KLK7, KLK11, NS3/4 protease of hepatitis C virus (HCV-NS3/4), tissue plasminogen activator (tPA), calpain, calpain 2, glutamate carboxypeptidase II, plasma kallikrein, AMSH-like protease, AMSH, 7-secretase component, antiplasmin cleaving enzyme (APCE), decysin 1, apoptosis-related cysteine peptidase, and N-acetylated alpha-linked acidic dipeptidase-like 1. In some embodiments, the linker can be cleaved by any one of these proteases. In some embodiments, the protease is MMP9. The cleavage or recognition site of each example protease is also known in the art.

In some embodiments, the protein constructs described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the protein construct has a tumor growth inhibition percentage that is less than 60%, 70%, 80%, 90%00, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100$$

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the protein constructs as described herein can effectively kill cells expressing the tumor antigen recognized by the protein construct. In some embodiments, at least 20%, 30%, 40%, 50%, 60%, 70%, 80% or 90% cells can be killed (e.g., with at least or about 30 nM, 20 nM, 10 nM, 5 nM, 3 nM, 1 nM, 100 pm, 10 pm, 1 pM, 100 fm, 10 fm, or 1 fm of the protein construct).

In some embodiments, the antibodies or antigen binding fragments in the protein construct have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments do not have a functional Fc region. For example, the antibodies or antigen binding fragments are Fab, Fab′, F(ab′)2, and Fv fragments.

The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

In some embodiments, the antibodies or antigen-binding fragments thereof comprises an Fc region that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc region is originated from an IgG antibody or antigen-binding fragment thereof. The sequences and differences of the IgG subclasses are known in the art, and are described, e.g., in Vidarsson, et al., Frontiers in Immunology 5 (2014); Irani, et al., Molecular Immunology 67.2 (2015): 171-182; Shakib, Farouk, ed., The human IgG subclasses: molecular analysis of structure, function and regulation. Elsevier, 2016; each of which is incorporated herein by reference in its entirety.

The present disclosure also provides a protein construct that includes an antibody or antigen-binding fragment thereof that cross-competes with any antibody or antigen-binding fragment as described herein. The cross-competing assay is known in the art, and is described e.g., in Moore et al., Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. MAbs. Vol. 5. No. 2. Taylor & Francis, 2013, which is incorporated herein reference in its entirety. The masking peptides as described herein can be used for an antigen binding domain that cross competes with any antibody as described herein.

In some embodiments, the antibody or antigen-binding fragment thereof comprises an Fc region. In some embodiments, the antibody or antigen-binding fragment thereof does not comprise an Fc region, e.g., as a single-domain antibody. In some embodiments, the antibody or antigen-binding fragment thereof of the present disclosure can be modified in the Fc region to provide desired effector functions or serum half-life.

In some embodiments, the protein construct described herein is a multi-specific antibody. In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety. In some embodiments, the first Fc region (e.g., the first CH3 domain in the Fc region) can include a tryptophan (Trp) at position 366 according to EU numbering; and the second Fc region (e.g., the other CH3 domain in the Fc region) can include one or more of the following a serine (Ser) at position 366, an alanine (Ala) at position 368, and/or a valine (Val) at position 407 according to EU numbering.

In some embodiments, the Fc region is derived from human IgG1, human IgG2, human IgG3, or human IgG4. In some embodiments, the antibodies or antigen binding fragments do not have a functional Fc region. In some embodiments, the Fc region has LALA mutations (L234A and L235A mutations in EU numbering), or LALA-PG mutations (L234A, L235A, P329G mutations in EU numbering).

Fragments of antibodies are suitable for use in the methods provided so long as they retain the desired affinity and specificity of the full-length antibody. Thus, a fragment of an antibody that binds to a human CD3 will retain an ability to bind to a human CD3. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or scFv antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

In some embodiments, the protein construct is a bi-specific antibody. Bi-specific antibodies can be made by engineering the interface between a pair of antibody molecules to maximize the percentage of heterodimers that are recovered from recombinant cell culture.

For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety. In some embodiments, the bispecific antibody is a bi-specific T-cell engager (BiTE). BiTEs are fusion proteins consisting of two single-chain variable fragments (scFvs) of different antibodies, or amino acid sequences from four different genes, on a single peptide chain of about 55 KD. One of the scFvs binds to T cells via the CD3 receptor, and the other to a tumor cell via a tumor specific molecule.

Also disclosed herein are protein constructs (e.g., “knobs-into-holes” T-cell engaging bi-specific antibodies), including (1) a first moiety comprising an amino acid sequence that is at least 80% identical to the sequence of SEQ ID NO: 3 or a portion thereof; (2) a second moiety comprising an antibody or antigen-binding fragment thereof that binds to human CD3, wherein the first moiety and the second moiety is linked via a linker; and (3) a third moiety that specifically binds to a tumor antigen.

In some embodiments, the heavy chain of the antibody or antigen-binding fragment of the second moiety or the third moiety further comprises at least one heavy chain constant region (CH). In some embodiments, the heavy chain constant region(s) comprises at least one mutation that reduces FcγR and complement binding. In some embodiments, the mutations comprises L234A and L235A; or T366S, L368A, and Y407V.

In some embodiments, the heavy chain of the antibody or antigen-binding fragment of the second moiety includes an amino sequence that is at least 80%, 85%, 90%, 95%, 96%, 97%, 98%, or 99% identical to SEQ ID NO: 57. In some embodiments, the heavy chain of the antibody or antigen-binding fragment of the third moiety includes an amino sequence that is at least 80%, 85%, 90%, 95%, 96%, 97%, 98%, or 99% identical to SEQ ID NO: 59.

In some embodiments, the light chain polypeptide of the antibody or antigen-binding fragment of the second moiety or the third moiety further comprises a light chain constant region (CL). In some embodiments, the light chain of the antibody or antigen-binding fragment of the second moiety includes an amino sequence that is at least 80%, 85%, 90%, 95%, 96%, 97%, 98%, or 99% identical to SEQ ID NO: 58.

The tumor antigen or tumor associated antigen targeted by the protein construct can be any suitable tumor antigen known in the art. Examples of tumor antigens include, but are not limited to, CD2, CD4, CD19, CD20, CD22, CD23, CD30, CD33, CD37, CD40, CD44v6, CD52, CD56, CD70, CD74, CD79a, CD80, CD98, CD138, EGFR (Epidermal growth factor receptor), VEGF (Vascular endothelial growth factor), VEGFR1 (Vascular endothelial growth factor receptor 1), PDGFR (Platelet-derived growth factor receptor), RANKL (Receptor activator of nuclear factor kappa-B ligand), GPNMB (Transmembrane glycoprotein Neuromedin B), EphA2 (Ephrin type-A receptor 2), PSMA (Prostate-specific membrane antigen), Cripto (Cryptic family protein 1B), EpCAM (Epithelial cell adhesion molecule), CTLA4 (Cytotoxic T-Lymphocyte Antigen 4), IGF-IR (Type 1 insulin-like growth factor receptor), GP3 (M13 bacteriophage), GP9 (Glycoprotein IX), CD42a, GP 40 (Glycoprotein 40 kDa), GPC3 (glypican-3), GPC1 (glypican-1), TRAILR1 (Tumor necrosis factor-related apoptosis-inducing ligand receptor 1), TRAILRII (Tumor necrosis factor-related apoptosis-inducing ligand receptor II), FAS (Type II transmembrane protein), PS (phosphatidyl serine) lipid, Muc (Mucin 1, PEM), Muc18, CD146, a501 integrin, α4β1 integrin, αv integrin (Vitronectin Receptor), Chondrolectin, CAIX (Carbonic anhydrase IX), GD2 gangloside, GD3 gangloside, GM1 gangloside, Lewis Y antigen, Mesothelin, HER2 (Human Epidermal Growth factor 2), HER3, HER4, FN14 (Fibroblast Growth Factor Inducible 14), CS1 (Cell surface glycoprotein, CD2 subset 1, CRACC, SLAMF7, CD319), 41BB CD137, SIP (Siah-1 Interacting Protein), CTGF (Connective tissue growth factor), HLADR (MHC class II cell surface receptor), PD-1 (Programmed Death 1, Type I membrane protein, PD-L1 (Programmed Death Ligand 1), PD-L2 (Programmed Death Ligand 2), IL-2 (Interleukin-2), IL-8 (Interleukin-8), IL-13 (Interleukin-13), PIGF (Phosphatidylinositol-glycan biosynthesis class F protein), NRP1 (Neuropilin-1), ICAM1, CD54, GC182 (Claudin 18.2), Claudin, HGF (Hepatocyte growth factor), CEA (Carcinoembryonic antigen), LTβR (lymphotoxin β receptor), Kappa Myeloma, Folate Receptor alpha, GRP78 (BIP, 78 kDa Glucose-regulated protein), A33 antigen, PSA (Prostate-specific antigen), CA 125 (Cancer antigen 125 or carbohydrate antigen 125), CA19.9, CA15.3, CA242, leptin, prolactin, osteopontin, IGF-II (Insulin-like growth factor 2), fascin, sPIgR (secreted chain of polymorphic immunoglobulin receptor), 14-3-3 eta protein, 5T4, ETA (epithelial tumor antigen), MAGE (Melanoma-associated antigen), MAPG (Melanoma-associated proteoglycan, NG2), vimentin, EPCA-1 (Early prostate cancer antigen-2), TAG-72 (Tumor-associated glycoprotein 72), factor VIII, Neprilysin (Membrane metallo-endopeptidase), 17-1 A (Epithelial cell surface antigen 17-1A), nucleolin, nucleophosmin, and any combination thereof. In some embodiments, the tumor antigen is CD20, PSA, PSCA, PD-L1, Her2, Her3, Her1, β-Catenin, CD19, CEACAM5, EGFR, c-Met, EPCAM, PSMA, CD40, MUC1, or IGF1R, etc.

In some embodiments, the tumor antigen is CEA or CEACAM5. In some embodiments, the moiety is an antibody or antigen-binding fragment thereof that binds to the tumor antigen. In some embodiments, the antibodies or antigen-binding fragments thereof is a single-chain variable fragment (scFv), a single-domain antibody (sdAb), or an antigen-binding fragment (Fab). In some embodiments, the antibodies or antigen-binding fragments thereof is further fused to a Fc domain. In some embodiments, the antibodies or antigen-binding fragments comprises a VHH. In some embodiments, the VHH comprises an amino sequence that is at least 80%, 85%, 90%, 95%, 96%, 97%, 98%, or 99% identical to SEQ ID NO: 52. In some embodiments, the amino acid sequences of the VHH CDR1, CDR2, and CDR3 are set forth in SEQ ID NOs: 53, 54 and 55, respectively. In some embodiments, the moiety targeting CEACAM5 has an amino acid sequence that is at least 80%, 85%, 90%, 95%, 96%, 97%, 98%, or 99% to SEQ ID NO: 59.

Any of the protein constructs described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of the protein constructs in vitro (e.g., in tissue culture or when stored as a pharmaceutical composition) or in vivo (e.g., in a human).

In some embodiments, the protein constructs described herein can be conjugated to a therapeutic agent. The protein-drug conjugate comprising the protein constructs can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Polynucleotides and Recombinant Vectors

The present disclosure also provides nucleic acid comprising a polynucleotide encoding the antibody masks, fusion peptides, and protein constructs described herein. The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

A vector is a construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition. The present disclosure provides a recombinant vector comprising a nucleic acid construct suitable for expressing the protein constructs, the fusion protein, the related antibodies or antigen binding fragments thereof.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein. In some embodiments, the viral vector contains an EF1α promoter to facilitate expression. In some embodiments, the vector is a lentivirus vector.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence.

Various cell lines can be used in connection with the vectors as described herein. Exemplary eukaryotic cells that may be used to express polypeptides include, but are not limited to, COS cells, including COS 7 cells; HEK293 cells, including HEK293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the protein constructs, antibodies or other related molecules. In one aspect, the disclosure relates to a cell comprising the vector or the pair of vectors as described herein.

In some embodiments, provided herein are vectors encoding the protein constructs, antibodies or other related molecules.

The present disclosure also provides a nucleic acid sequence comprising a nucleotide sequence encoding any of the protein constructs, antibodies or other related molecules (including e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure provides methods of inducing or activating a T cell immunity in a subject, comprising administering an effective amount of the protein construct described herein to the subject.

The induction of T cell immunity can be induced by the binding of a moiety of the protein construct, e.g., the anti-CD3 antigen binding domain to the CD3 on T cells (e.g., cytotoxicity T cells). In some embodiments, the T cell immunity is induced or activated upon the cleavage of the protease cleavable domain of the linker. When the moiety (e.g., the anti-CD3 antigen binding domain) interacts with the antibody mask, the binding of the anti-CD3 antigen binding domain to its target (e.g., CD3 on T cells) is blocked. Only upon the removal or dissociation of the antibody masks does the antigen binding domain bind to its target, therefore inducing the T cell activation. In some embodiments, the antibody masks are removed or dissociated by a protease and its digestion of the protease cleavable linker. Any suitable protease can be used in the methods described herein. In some embodiments, the protease is MMP9.

In one aspect, the disclosure features methods that include administering a therapeutically effective amount of the protein construct provided herein to a subject in need thereof (e.g., a subject having, or identified or diagnosed as having, a disease or disorder (e.g., cancer)). The cancer can be, for example, acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; lung cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilms' tumor.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

As used herein, the term “delaying development of a disease” refers to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, can be delayed.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

Atypical dosage of an effective amount of the protein construct can be 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the protein construct, optionally with at least one additional therapeutic agent, can be administered to the subject at least once a week (e.g., once a week, twice a week, three times a week, four times a week, once a day, twice a day, or three times a day). In some embodiments, protein constructs and at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation. In some embodiments, the one or more additional therapeutic agents can be administered to the subject prior to, concurrently with, or after administering the protein constructs to the subject.

In some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2, 3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX₃CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

In some embodiments, the subject is not responsive to the treatments including e.g., resection, radiation, ablation, chemoembolization, liver transplantation, targeted drug therapy (Kinase inhibitors: Sorafenib, lenvatinib, Regorafenib, Cabozantinib) and some immune checkpoint inhibitors. In some embodiments, one or more of these treatments are administered to the subject in combination with the protein constructs described herein.

### Compositions and Formulations

The present disclosure provides compositions (including pharmaceutical and therapeutic compositions) containing the protein constructs produced by the methods disclosed herein. Also provided are methods, e.g., therapeutic methods for administrating the protein constructs and compositions thereof to subjects, e.g., patients or animal models (e.g., mice).

The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular protein constructs and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the protein constructs, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/orvincristine.

The pharmaceutical composition in some embodiments contains the protein constructs in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the protein constructs, by multiple bolus administrations of the protein constructs, or by continuous infusion administration of the protein constructs.

The compositions can be administered using standard administration techniques, formulations, and/or devices. Administration of the compositions can be autologous or heterologous.

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The compositions or pharmaceutical compositions as described herein can be included in a container, pack, or dispenser together with instructions for administration.

Provided are also methods of administering the compositions, and uses of such compositions to treat or prevent diseases, conditions, and disorders, including cancers. In some embodiments, the methods described herein can reduce the risk of the developing diseases, conditions, and disorders as described herein.

In some embodiments, the compositions described herein are administered to a subject or patient having a particular disease or condition to be treated. In some embodiments, compositions prepared by the provided methods are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the protein constructs.

In some embodiments, the subject has been treated with a therapeutic agent targeting the disease or condition, e.g. the tumor, prior to administration of the composition described herein. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

The compositions described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the compositions. In some embodiments, it is administered by multiple bolus administrations of the compositions, for example, over a period of no more than 3 days, or by continuous infusion administration of the compositions.

### EXAMPLES

The examples provided below are for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1. Production of Antibodies and Proteins

SP34 is an antibody targeting human CD3 complex (see, e.g., Pessano et al. The EMBO Journal. 4:337-344, 1985). It was shown to bind to the s domain of CD3 (see, e.g., A Salmerón et al. J. Immunology. 147:3047-3052, 1991). Due to its cross-reactivity to CD3 of a variety of non-human primates (see, e.g., Conrad et al. Cytometry A. 71:923-933, 2007), its fragment, either in Fab or scFv format, was often employed as the anti-CD3 antibody arm in T cell engager (TCE) bispecific antibodies.

The aim of this study is to identify peptides that can block the binding of SP34 to CD3 and use such peptide as a mask to disable the binding of the antibody. Fusion of such a masking peptide to SP34 through a protease-digestible linker would potentially generate a masked TCE (M-TCE) which is only active in a microenvironment where the proteinase is abundant.

All generated molecules including SP34, peptide-Fc fusions and bispecific antibodies (bsAbs) were produced in a transient expression system using HEK293F as the expression host. Briefly, 0.3×10⁶ 293F cells were used to inoculate 300 mL KOP293 medium (Zhuhai Kairui, Zhuhai, China) and allowed for cell proliferation for 2-4 days with 120 rpm in 5% CO₂. Next, the cells were used to inoculate 100 mL of KPM Transfection Medium (Zhuhai Kairui, Zhuhai, China) to reach a cell density of 1×10⁶ cells/mL, and allowed for growth for overnight. 100 μg plasmid DNA encoding the designed constructs were mixed with 600 g polyethylenimine (PEI) by slowly adding the PEI solution to the plasmid solution. After incubation at room temperature (RT) for 15 minutes, the plasmid-PEI mixture was added to the HEK293F cells, and the cells were cultured as described above. The cells were allowed for growth for 7 days. 2 mL KT-feed (50×, Zhuhai Kairui) was added after 24 hours of growth. Harvest of culture supernatant was conducted by centrifugation and filtration through a 0.22 m filter. Antibodies were purified by Protein A affinity chromatography using MabSelect™ PrismA FF column (GE) following manufacturer's instructions.

### Example 2. Binding of Antibodies and Masked Antibodies to their Target

Bio-Layer Interferometry (BLI) technique was employed for the measurement of protein-protein interactions in this study. A Octet Red96 system and Streptavidin (SA) or AHC biosensors (ForteBio, Fremont) were utilized.

To measure the binding of SP34 to CD3ε peptides, the peptides were immobilized on the SA biosensor through SA-biotin interaction according the manufacturer's instruction. After running PBS with 0.01% Tween-20 buffer to stabilize baselines, the biosensors were dipped into solid black 96-well plates (Greiner Bio-One, Cat. #65520, Monroe, North Carolina, USA) containing SP34 in indicated concentrations. Association and dissociation were measured accordingly, and binding kinetics were calculated using ForteBio's Data Analysis 12.0 software.

Measurement of bindings between CD3ε₁₋₂₇ mutants-SP34 fusion, or M-TCE, and CD36s was conducted as described above but with AHC sensors to capture M-TCE, and CD36s was used as the analyte.

### Example 3. Determination of Minimum Length of N-Terminal Peptide of CD3ε for its SP34 Binding

SP34 binds to an epitope located at the N-terminus of CD3ε. We sought to use peptides and mutants of the natural epitope of SP34 as its own mask.

### Measurement of the Affinity of SP34 to CD3δε

Gene coding for VH and V_{L} of SP34 (SEQ ID NOs: 1 and 2, respectively) was synthesized at GenScript Inc. and fused to the constant regions of human IgG1 to generate SP34 chimeric antibody, which was produced as described above.

CD3δε was purchased from ACRO (Beijing, China) and used as the antigen for SP34 affinity measurement.

SP34 was immobilized on the AHC biosensor and its affinity to CD3δε was measured using the latter as the analyte. This approach was selected as it can avoid potential multivalent binding and give accurate affinity between SP34 and CD3δε.

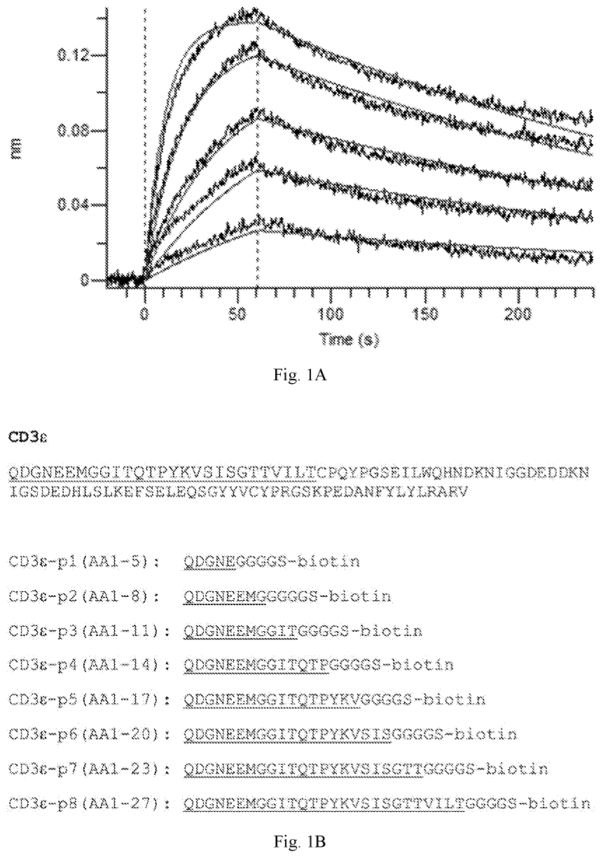

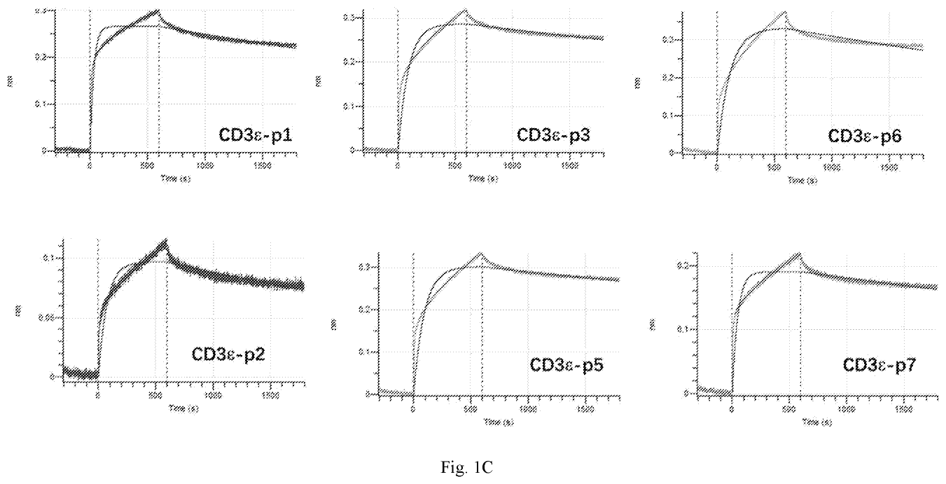

With multiple CD3δε concentrations (9.4, 18.8, 37.5, 75.0 and 150 nM) applied, the affinity of monovalent binding of SP34 was determined as 5.8×10⁻⁹ M (FIG. 1A, Table 1).

TABLE 1
Affinities of SP34 to CD3δε and various
N-terminal peptides of CD3ε

| Sample ID | kₐ (1/Ms) | k_{d} (1/s) | K_{D} (M) |
|---|---|---|---|
| CD3δε | 5.5 × 10⁵ | 3.2 × 10⁻³ | 5.8 × 10⁻⁹ |
| CD3ε-p1 | 1.6 × 10⁵ | 1.6 × 10⁻⁴ | 9.9 × 10⁻¹⁰ |
| CD3ε-p2 | 6.5 × 10⁴ | 2.4 × 10⁻⁴ | 3.6 × 10⁻⁹ |
| CD3ε-p3 | 5.6 × 10⁴ | 1.1 × 10⁻⁴ | 2.0 × 10⁻⁹ |
| CD3ε-p5 | 5.4 × 10⁴ | 9.8 × 10⁻⁵ | 1.8 × 10⁻⁹ |
| CD3ε-p6 | 4.6 × 10⁴ | 1.6 × 10⁻⁴ | 3.5 × 10⁻⁹ |
| CD3ε-p7 | 1.0 × 10⁵ | 1.3 × 10⁻⁴ | 1.3 × 10⁻⁹ |

### Measurement of the Affinity of SP34 to N-Terminal Peptides of CD3ε

The sequence of human CD3ε peptides (SEQ ID NO: 3) is shown in FIG. 1B. Eight peptides ranging from 5 AA to 27 AA of its N-terminus with a GGGGS linker (SEQ ID NOs: 4-11) and a biotinylation at their C-terminus were synthesized at Sangon Biotech (Shanghai, China), among which six (CD3ε-p1, p2, p3, p5, p6 and p7) were obtained.

BLI technology was employed to measure the affinity of the six peptides to SP34 with a ForteBio Red96. These peptides were immobilized on the SA Biosensor and their affinities were measured using SP34 as the analyte. The binding sensorgrams indicate that SP34 binds to all six peptides with very similar profiles (FIG. 1C). The affinities of the bindings were determined as between 1-4 nM (Table 1), very similar to that of CD3δε/SP34 binding. This result clearly demonstrates that a N-terminal CD3ε peptide as short as five amino acids binds to SP34 almost as strong as CD3δε does. The results also demonstrated that N-terminal CD3ε peptides with lengths of 5 AA, 8 AA, 11 AA, 17 AA, 20 AA and 23 AA are all capable of binding to SP34. The results at least demonstrated that any N-terminal CD3ε peptide in a length between at least 5 AA to 23 AA is capable of binding to SP34. It is further hypothesized that a N-terminal CD3ε peptide shorter than 5 AA and longer than 23 AA can bind to SP34 as well.

### Example 4. Screening of CD3ε Mutant Peptides that Bind SP34

Generation of a CD3ε₁₋₂₇-Fc fusion

We next made a monovalent fusion of AA1-27 of CD3ε (SEQ ID NO: 67): QDGNEEMGGITQTPYKVSISGTTVILT) to human Fc by fusing the peptide through a peptide linker IEGRMD to the human IgG4 hinge+Fc region of the knob chain of a knob-in-hole construct while leaving the hole chain of the Fc unchanged. The fusion protein was produced as described above.

Examination of binding of SP34 to CD3ε₁₋₂₇-Fc fusion proteins was conducted by ELISA. 100 μL of 2 μg/mL fusion protein was coated on 96 well microtiter plate (Nunc) at room temperature (RT) overnight. After blocking the wells with 3% milk-PBS (3% non-fat milk in 137 mM NaCl, 2.7 mM KCl, 10 mM Na₂HPO₄ 1.8 mM KH₂PO₄) at RT for 1 hr and 6 washes with 0.05% PBST (0.05% Tween-20 in PBS), SP34 (5 μg/mL, 50 μL/well) was added to the wells as the first antibody and allowed for incubation at 37° C. for 1 hr. The wells were washed for 6 times with PBST after the incubation, and the second antibody horseradish peroxidase (HRP) labeled anti-Kappa light chain (Sigma, 1:6,000 dilution, 50 μL/well) was added and allowed for incubation at RT for 50 min. After 6 washes with PBST, 3,3′, 5,5′-Tetramethylbenzidine (TMB) was added to develop color, and 1M HCl was then added to stop the reaction. ELISA titer was read with a microtiter plate reader (Thermo Fisher) at 450 nm wavelength.

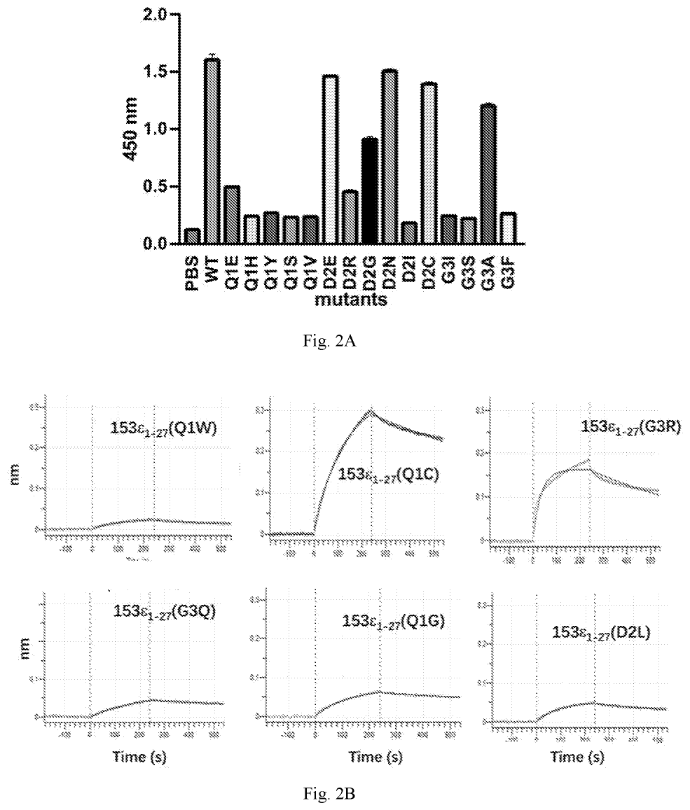

As shown in FIG. 2A, SP34 demonstrated strong binding to CD3ε₁₋₂₇-Fc fusion. We then introduced mutations in this peptide and screened for mutants that maintained binding of the peptide to SP34.

Screening of CD3 [symbol] ₁₋₂₇-Fc Mutants that Maintained Binding to SP34

Each of the first three amino acids (QDG) of the peptide in the CD3 [symbol] ₁₋₂₇-Fc fusion protein was mutated to the other 19 possible ones. This effort generated 57 mutant fusion proteins, all were produced as described above.

In some embodiment, binding of SP34 to the first batch of 15 variants of the CD3 [symbol] ₁₋₂₇ mutant fusion proteins were tested by ELISA as described above. Among the 15 variants, five mutants (D2E, D2G, D2N, D2C and G3A) showed significant binding to SP34 (FIG. 2A).

In some embodiment, binding of SP34 to the second batch of 42 variants of CD3 [symbol] ₁₋₂₇ mutant fusion proteins were tested by BLI with a ForteBio Red96 as described above. The majority of the variants demonstrated largely reduced binding to SP34 (FIG. 2B, only six out of the 42 representative sensorgrams are shown). Among the 42 variants, two mutants Q1C and G3R showed similar binding profiles as that of the wild type.

In brief summary, seven mutants (Q1C, D2E, D2G, D2N, D2C, G3A and G3R) of the CD3 [symbol] ₁₋₂₇-Fc fusion proteins maintained binding to SP34, and CD3: peptides containing these mutations were therefore considered potential masks of SP34.

### Example 5. Construction and Characterization of Masked T Cell Engagers (M-TCE) Based on SP34-CD3: Peptide Fusions

### Construction and Generation of Masked Antibodies

With SP34 as the anti-CD3 antibody and seven potential masks identified above, we next constructed variants of M-TCEs. An anti-CEACAM5 single domain antibody (see, e.g., PCT Publication No. WO2019135159), designated CEA, was selected as a model anti-TAA antibody.

It is predicted that these M-TCEs would lose or have reduced affinity to CD3. It was further devised that the M-TCEs would be activated upon removal of the mask from the antibody by a protease. It is known that certain proteases have substantially higher level concentrations in tumor tissues in comparison to normal organs. Our design would render a non-functional M-TCE functional or make a less functional M-TCE more functional in an environment where the M-TCE can be activated by a specific protease. Ideally such a protease has high abundance in tumor but low abundance in normal organs, and the M-TCE can be activated only at tumor sites.

Matrix metallopeptidase 9 (MMP9) was selected as a model protease as it is associated with tumor progression of multiple types of cancer.

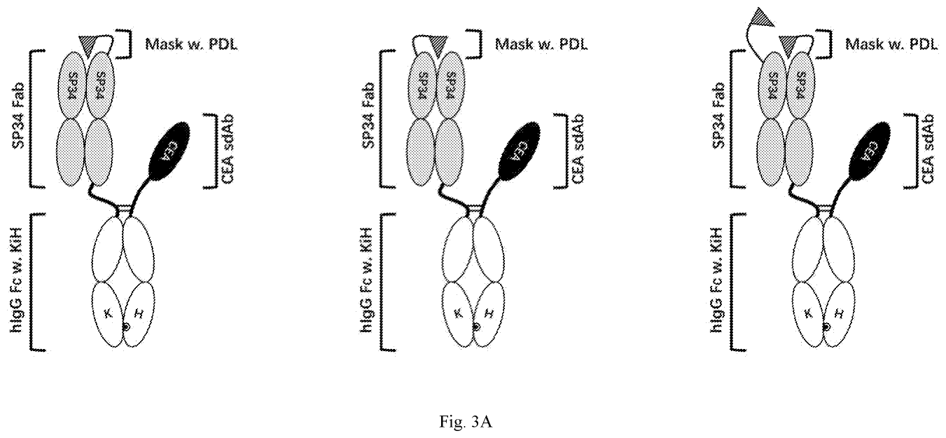

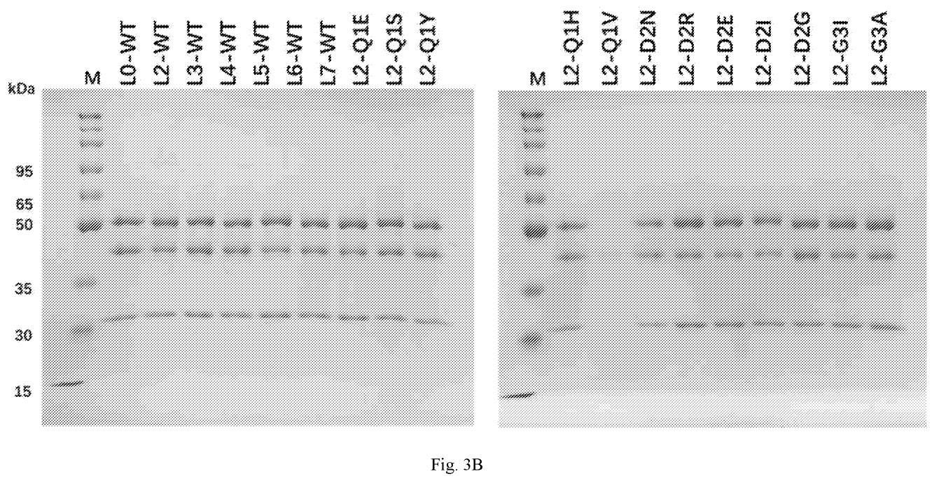

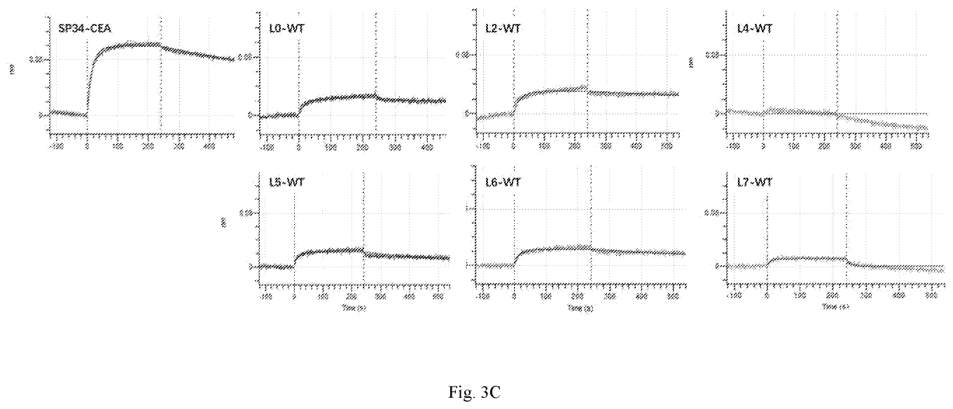

Our designed M-TCEs have the following components (FIG. 3A): i) a Fc region with knob-in-hole (KiH)-enabling mutations; ii) a TAA binding antibody, either a Fab, an scFv or an sdAb, constructed on either the knob or the hole chain of the Fc; In the FIG. 3A diagram, the anti-CEACAM5 sdAb CEA was placed to the hole chain of the Fc; iii) Fab of an anti-CD3 antibody, e.g., SP34, which binds to an epitope QDGNEE or variants identified in this study constructed on the opposite chain of the TAA antibody fragment; iv) a mask for the anti-CD3 antibody linked by v) a protease digestible linker (PDL) to the N-terminus of either the V_{H} or the V_{L} of the antibody.

It is noteworthy that the masks can be put on the heavy chain, the light chain, or even both chains of the an anti-CD3 antibody, as illustrated in the left, middle and right panel, respectively.

It was demonstrated above that a five amino acid peptide of the N-terminus CD3 bound to SP34 with similar affinity as CD38s. Six amino acid peptides, either in their wild type sequence of QDGNEE or mutations identified above, were employed as potential masks. The mutations of the hexapeptide QDGNEE (SEQ ID NO: 68) used included D2E, D2G, D2N G3A and G3R. Q1C and D2C mutations were not pursued further, because a free cysteine is usually not preferable in a protein construct.

In this study a peptide PLGL was selected as the protease digestion sequence. This sequence can be readily digested by proteases such as MMP9. In some constructs the mask sequence was directly fused to the PDL and then directly fused to the SP34 VH. In some constructs linkers are inserted between the mask and the PDL and also between the PDL and the SP34 VH. These different constructs serve the purpose of finding optimal linker length between the mask and the antibody, as this length will likely have a big impact on the stability of a M-TCE.

Sequences of mask+PDL part of all constructed M-TCE are listed in Table 2.

TABLE 2
Potential masks of SP34 with their connecting linkers used in this study

| | Position | | Masking | Experimental | SEQ ID |
|---|---|---|---|---|---|
| Name | of masks | Masks and linkers | Capability | procedure | NO |
| L0-WT | V_{H} | QDGNEEPLGL-SP34V_{H} | Yes | ELISA, BLI | 12 |
| L2-WT | V_{H} | QDGNEEGSPLGLGS-SP34V_{H} | Yes | ELISA, BLI | 13 |
| L3-WT | V_{H} | QDGNEESGSPLGLSGG-SP34V_{H} | Yes | ELISA, BLI | 14 |
| L4-WT | V_{H} | QDGNEEGSGSPLGLGSGS-SP34V_{H} | Yes | ELISA, BLI | 15 |
| L5-WT | V_{H} | QDGNEESSGGSPLGLSSGGS-SP34V_{H} | Yes | ELISA, BLI | 16 |
| L6-WT | V_{H} | QDGNEESSGGGSPLGLSSGGGS-SP34V_{H} | Yes | ELISA, BLI | 17 |
| L7-WT | V_{H} | QDGNEESSGGSGSPLGLSSGGSGS-SP34V_{H} | Yes | ELISA, BLI | 18 |
| L2-Q1E | V_{H} | EDGNEEGSPLGLGS-SP34V_{H} | No | ELISA, BLI | 19 |
| L2-Q1S | V_{H} | SDGNEEGSPLGLGS-SP34V_{H} | No | ELISA, BLI | 20 |
| L2-Q1Y | V_{H} | YDGNEEGSPLGLGS-SP34V_{H} | No | ELISA, BLI | 21 |
| L2-Q1H | V_{H} | HDGNEEGSPLGLGS-SP34V_{H} | No | ELISA, BLI | 22 |
| L2-Q1V | V_{H} | VDGNEEGSPLGLGS-SP34V_{H} | No | ELISA, BLI | 23 |
| L2-D2N | V_{H} | QNGNEEGSPLGLGS-SP34V_{H} | n.a. | ELISA, BLI | 24 |
| L2-D2R | V_{H} | QRGNEEGSPLGLGS-SP34V_{H} | No | ELISA, BLI | 25 |
| L2-D2E | V_{H} | QEGNEEGSPLGLGS-SP34V_{H} | Yes | ELISA, BLI | 26 |
| L4-D2E | V_{H} | QEGNEEGSGSPLGLGSGS-SP34V_{H} | Yes | BLI | 27 |
| L5-D2E | V_{H} | QEGNEESSGGSPLGLSSGGS-SP34V_{H} | Yes | BLI | 28 |
| L6-D2E | V_{H} | QEGNEESSGGGSPLGLSSGGGS-SP34V_{H} | Yes | BLI | 29 |
| L7-D2E | V_{H} | QEGNEESSGGSGSPLGLSSGGSGS-SP34V_{H} | Yes | BLI | 30 |
| L2-D2I | V_{H} | QIGNEEGSPLGLGS-SP34V_{H} | No | ELISA, BLI | 31 |
| L2-D2G | V_{H} | QGGNEEGSPLGLGS-SP34V_{H} | Yes | ELISA, BLI | 32 |
| L4-D2G | V_{H} | QGGNEEGSGSPLGLGSGS-SP34V_{H} | Yes | BLI | 33 |
| L5-D2G | V_{H} | QGGNEESSGGSPLGLSSGGS-SP34V_{H} | Yes | BLI | 34 |
| L6-D2G | V_{H} | QGGNEESSGGGSPLGLSSGGGS-SP34V_{H} | Yes | BLI | 35 |
| L7-D2G | V_{H} | QGGNEESSGGSGSPLGLSSGGSGS-SP34V_{H} | Yes | BLI | 36 |
| L2-G3I | V_{H} | QDINEEGSPLGLGS-SP34V_{H} | No | ELISA, BLI | 37 |
| L2-G3A | V_{H} | QDANEEGSPLGLGS-SP34V_{H} | Yes | ELISA, BLI | 38 |
| L4-G3A | V_{H} | QDANEEGSGSPLGLGSGS-SP34V_{H} | Yes | BLI | 39 |
| L5-G3A | V_{H} | QDANEESSGGSPLGLSSGGS-SP34V_{H} | Yes | BLI | 40 |
| L6-G3A | V_{H} | QDANEESSGGGSPLGLSSGGGS-SP34V_{H} | Yes | BLI | 41 |
| L7-G3A | V_{H} | QDANEESSGGSGSPLGLSSGGSGS-SP34V_{H} | Yes | BLI | 42 |
| L2-G3R | V_{H} | QDRNEEGSPLGLGS-SP34V_{H} | Yes | BLI | 43 |
| L4-G3R | V_{H} | QDRNEEGSGSPLGLGSGS-SP34V_{H} | Yes | BLI | 44 |
| L6-G3R | V_{H} | QDRNEESSGGGSPLGLSSGGGS-SP34V_{H} | Yes | BLI | 45 |
| LC-L4- | V_{L} | QEGNEEGSGSPLGLGSGS-SP34V_{L} | Yes | BLI | 27 |
| G2E | | | | | |
| 2XL4- | V_{H} and | QEGNEEGSGSPLGLGSGS-SP34V_{H} | Yes | BLI | 27 |
| G2e | V_{L} | QEGNEEEGSGSPLGLGSGS-SP34V_{L} | | | |

*n.a.: no available.

The first batch of 19 constructs included seven M-TCEs with a mask sequence of QDGNEE, the wildtype hexapeptide of CD3: N-terminus, with PDL lengths of 4, 8, 10, 12, 14, 16 and 18 AA, respectively, and 12 M-TCEs with 12 potential mutant masks with a PDL length of 8 AA (Table 2).

All M-TCE could be successfully produced in our 293 transient expression system (FIG. 3B). Lack of visible protein bands in lane Q1V of FIG. 3B was due to a sample loading error.

### Characterization of Masked Antibodies

Binding of the 19 M-TCEs to both their antigens CEACAM5 and CD3δε was assessed with BLI using ForteBio Red96 as described above.

As expected, all 19 M-TCEs and the non-masked TCE antibody control SP34-CEA bound to CEACAM5 with very similar binding profiles (data not shown), suggesting that masking of SP34 does not interfere with binding of the anti-TAA antibody to its antigen.

The non-masked TCE antibody control bound CD3δε as expected (FIG. 3C, upper left panel). All M-TCE with the wild type mask (SEQ ID NO: 68: QDGNEE) displayed much reduced binding in contrast to the control TCE. This result suggests that the linker length between the mask and the antibody is widely adjustable without influencing the binding of the mask to SP34, and linker length of at least 8-18 AA is acceptable.

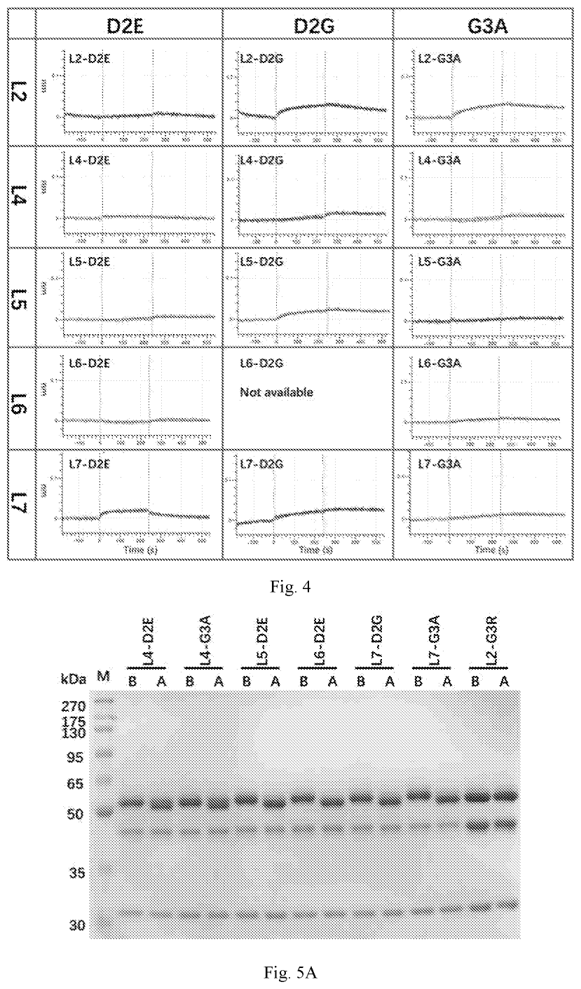

When the putative masks were linked to SP34 with an eight amino acid linker (e.g., L2: GSPLGLGS, SEQ ID NO: 86), M-TCEs with three masks different from the wild type mask QDGNEE (SEQ ID NO: 68), i.e., D2E mutant (QEGNEE, SEQ ID NO: 76), D2G mutant (QGGNEE, SEQ ID NO: 78) and G3A mutant (QDANEE, SEQ ID NO: 80) that showed binding to SP34 (FIG. 2) demonstrated reduced binding to CD3δε (FIG. 4, upper panel) in comparison to the unmasked TCE SP34-CEA (FIG. 3C). This result demonstrated that the three peptides are as effective as the wild type mask QDGNEE (SEQ ID NO: 68), if not more effective, in blocking the binding of SP34 to CD3.

M-TCE with the seven mutants that were shown to have low or no binding to SP34 (FIG. 2A) displayed similar binding profiles as of non-masked TCE (Data not shown). These mutants were unlikely to become efficient masks and were not exploited further.

In the second batch more M-TCE constructs were generated and tested.

As for the masks, D2E, D2G and G3A mutants of QDGNEE (SEQ ID NO: 68) were tested.

As for the linker length, five linkers (L2, L4, L5, L6 and L7, representing total linker length of 8, 12, 14, 16 and 18 AA) were used.

Combination of the mask mutations and the linker lengths gave rise to 15 M-TCEs (Table 2), of which 14 were successfully produced.

We first examined the binding of the M-TCEs to CD3δε (FIG. 4). All generated M-TCEs with mutations D2E, D2G and G3A demonstrated substantially lower binding to the antigen in comparison to non-masked TCE. These results suggested that linker lengths could vary from 8 to 18 AA in the M-TCE without greatly affecting the masking capability of the mask. Similar result was also observed for the wild type mask QDGNEE. Even longer or shorter linker lengths may function as well, which needs to be tested.

### Removal of Masks by Protease Digestion

Seven M-TCEs were selected for further analysis.

MMP9 proenzyme (proMMP9), purchased from Acrobiosystems, was dissolved to 100 μg/mL in MMP9 digestion buffer (50 mM Tris, 10 mM CaCl₂), 150 mM NaCl, pH 7.6). 4-aminophenylmercuric acetate (APMA) was added to a final concentration of 1 mM. The proMMP9-APMA mixture was incubated at 37° C. for 24 hr to activate proMMP9.

The selected M-TCEs, after adjusting to MMP9 digestion buffer, was mixed with activated MMP9 to a final concentration of 100 nM. The samples were further incubated at 37° C. for 3 hr to allow M-TCEs to be digested by MMP9.

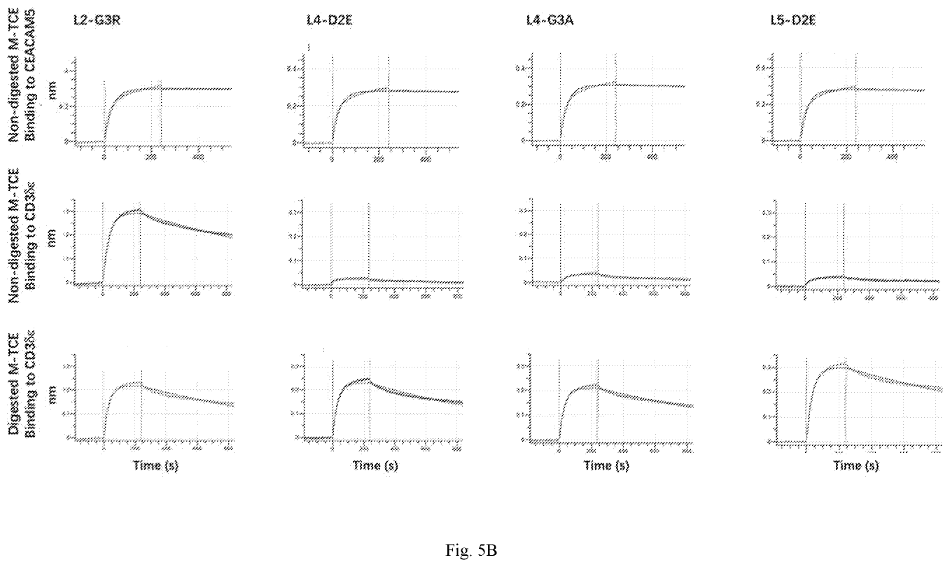

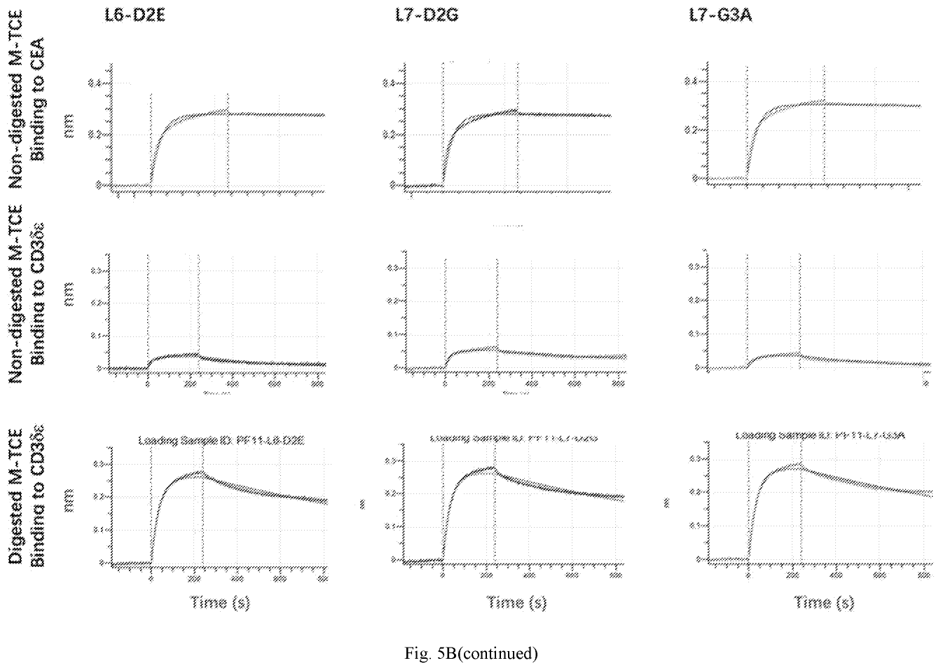

Removal of the masks from the M-TCEs was first revealed by SDS-PAGE (FIG. 5A). The M-TCE with an 8 AA linker, L2-G3R, could not be digested under the digestion condition, as no difference in the migration pattern was observed before and after the digestion. The two M-TCEs with a 12 AA linker, L4-D2E and L4-G3A, could be partially digested as faster migrating bands were visible, yet a small portion of the original bands could still be seen. The M-TCEs with 14 AA, 16 AA and 18 AA linker could be completely or nearly completely digested, as only the faster migrating bands could be seen. These results suggest that linker length of 14 to 18 are appropriate for not only blocking the binding of SP34 by masks identified in this study but also for easy removal of the mask by protease digestion. Too short linker would make the removal of the masks difficult, probably because the employed enzyme, MMP9 in this case, could not fully engage with its digestion site in the M-TCEs.

Undigested and digested M-TCEs were further analyzed by BLI for their binding to both CEACAM5 and CD3δε (FIG. 5B). As expected, all proteins bound to CEACAM5 with similar binding profiles before MMP9 digestion (FIG. 5B, upper panel), indicating that binding of the sdAb arm of the M-TCE was not influenced by the masks.

All but one (L2-G3R) M-TCEs demonstrated much reduced binding to CD3δε (FIG. 5B, middle panel). Given the fact that G3R mutant of the wild type mask, i.e., QDRNEE (SEQ ID NO: 83), was able to bind SP34, this lack of masking capability may be caused by the linker connecting it to SP34. It is possible that the G3R mask could block the binding of SP34 if a different linker is used.

Masks of the rest of the M-TCEs could be successfully removed. After digestion of the M-TCEs by MMP9, their binding capability to CD3δε was largely recovered (FIG. 5B, lower panel), indicating successful removal of the masks. This result further confirmed the SDS-PAGE analysis of the digestions (FIG. 5A).

### Efficacy of M-TCE Before and After Mask Removal

Five digested and undigested M-TCEs were tested for their tumor cell killing capability in the presence of human PBMC.

### PBMC Co-Cultivation Assays

A total of 5×10³ cells in RPMI1640 medium (supplemented with 10% FBS and 2 mM glutamine) of the target cell line (HT29) were seeded in 96-well plates (Day 0). A dilution series of respective antibodies was performed in assay media and added to target cells. On Day 0, a total of 5×10⁴ PBMCs were added to each well, and the final reaction volume was adjusted to 200 μl. Percentage viability of target cell was measured by lactate dehydrogenase (LDH) release of live cells. LDH was measured after 72 hours using the Cell Counting Kit-8 (CK04, Dojindo, Japan) according to the manufacturer's recommendations. The results were analyzed as mean and standard deviation (SD) from triplicate wells and plotted as 4-parameter non-linear regression fittings using GraphPad Prism 9 software (GraphPad Software, San Diego, CA, USA).

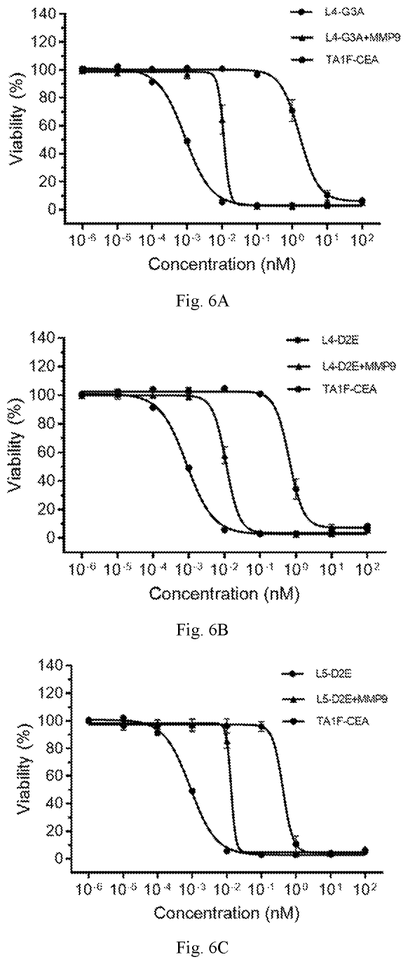

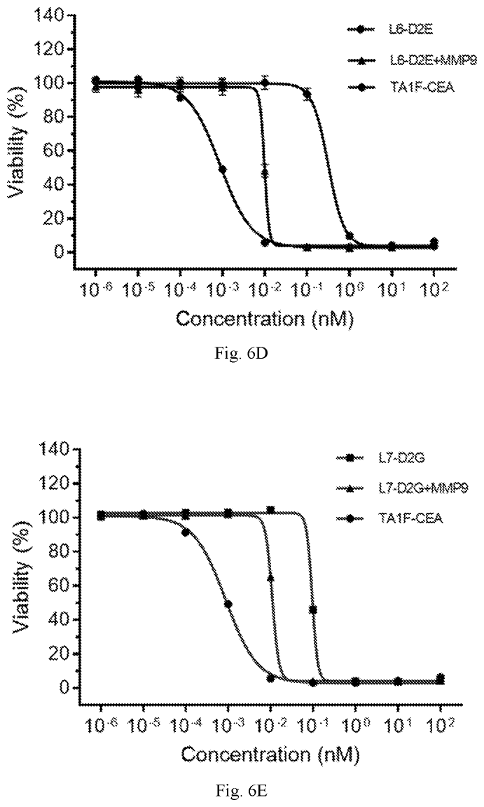

FIG. 6 is a representative result of three similar experiments. The L4-G3A M-TCE could induce tumor cell killing both before and after MMP9 digestion, yet the efficacy was ˜140 fold lower before mask removal (FIG. 6, Table 3). It is noteworthy that removal of the mask could not fully restore the activity of M-TCE in comparison to unmasked TCE, SP34-CEA: EC50 of MMP9-digested M-TCE was approximately 10 fold of that of unmasked TCE.

TABLE 3
Efficacy (EC₅₀) of digested and undigested M-TCEs

| | L4-D2E | L4-G3A | L5-D2E | L6-D2E | L7-D2G |
|---|---|---|---|---|---|
| Unmasked | 1 pM | | | | |
| Non-digested | 650 pM | 1722 pM | 418 pM | 317 pM | 96 pM |
| MMP9-digested | 11 pM | 12 pM | 13 pM | 10 pM | 11 pM |
| Digested vs. masked | 59× | 144× | 32× | 32× | 9× |

Similar results were also seen from other M-TCEs including L4-D2E, L5-D2E, L6-D2E and L7-D2G. In these cases all MMP9-digested M-TCE demonstrated higher efficacy in inducing tumor cell killing than their undigested counterparts, yet their efficacy were not as high as that of the unmasked TCE.

### Characterization of Heavy Chain Mask, Light Chain Mask and Double Masks

As illustrated in FIG. 3A, M-TCE can be potentially generated by fusing a mask on the heavy chain, the light chain or even both chains of SP34. To evaluate the feasibility of chain selection freedom, a mask G3A (QDANEE) was fused to the VH, the VL and both VH and VL of SP34 TCE through one (for single mask) or two (for double mask) 12 AA linker L4 (GSGSPLGLGSGS, SEQ ID NO: 88). Three resulting M-TCEs, SP34-L4-G3A, SP34-LC-L4-G3A and SP34-2XL4-G3A, together with a non-masked control SP34-CEA, were produced and characterized.

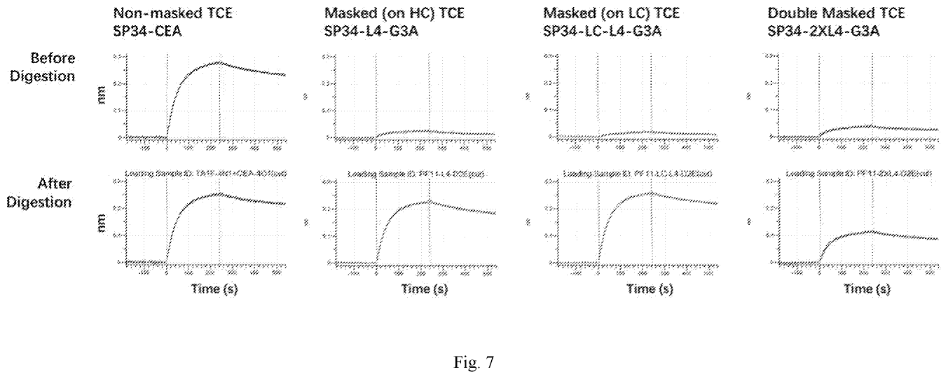

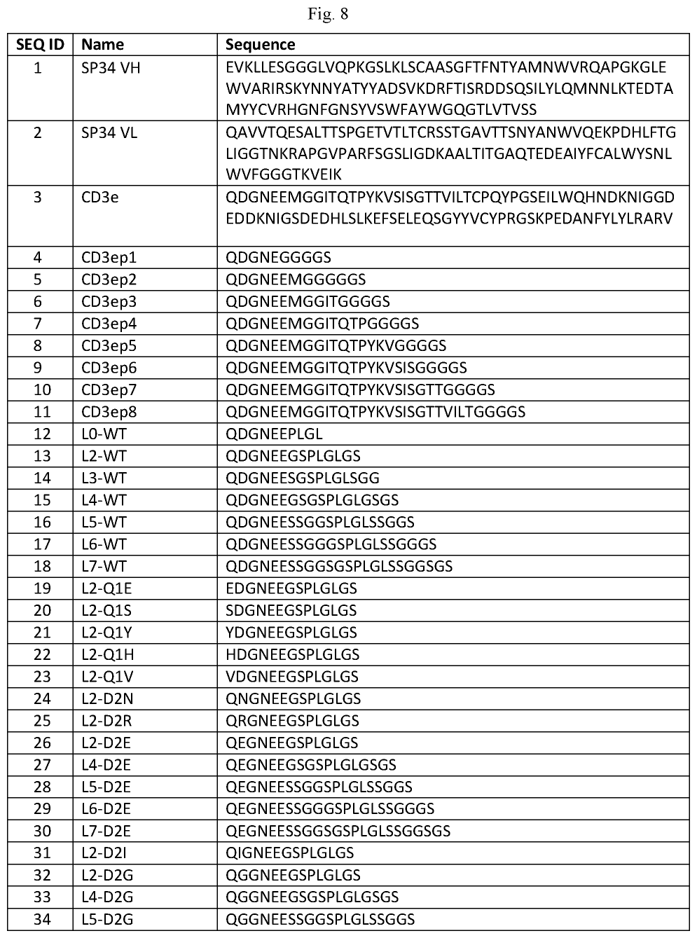

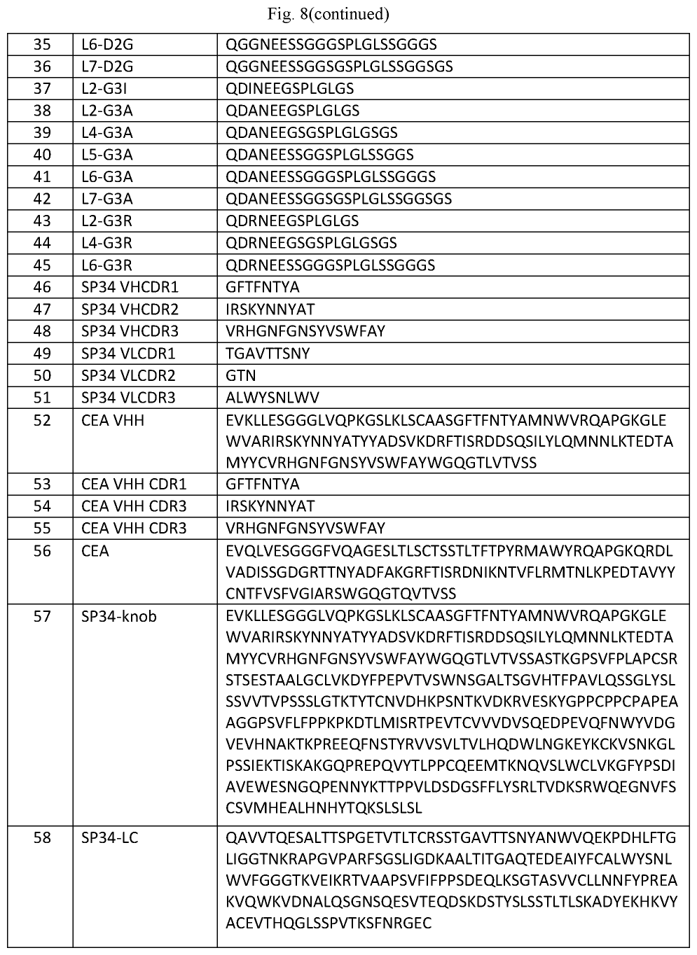

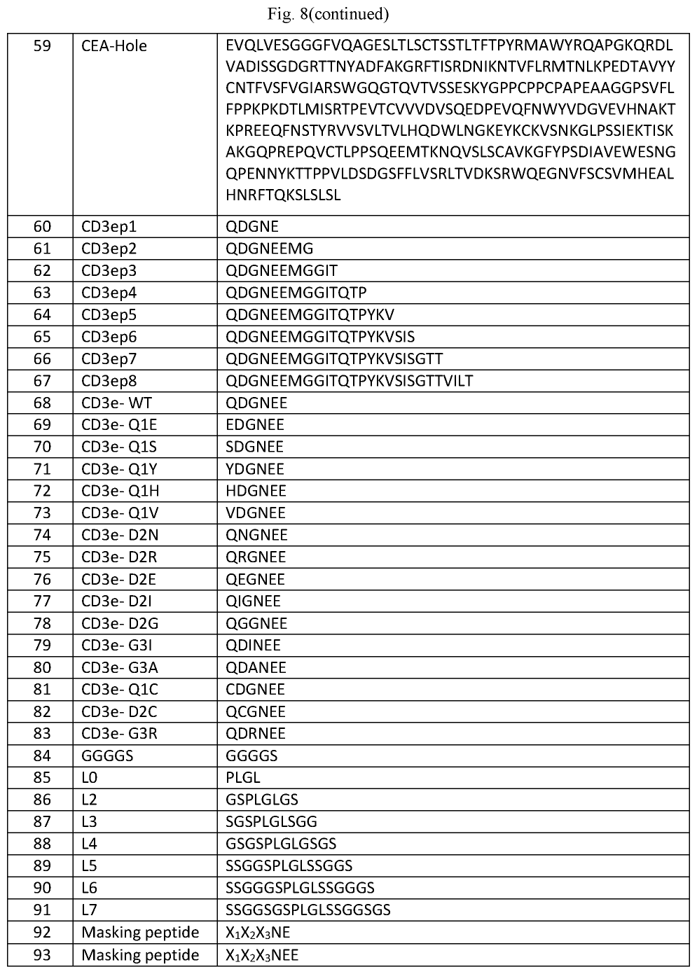

The proteins were digested by MMP9 with the aforementioned method and analyzed by measuring the binding of digested and non-digested proteins to CD3δε (FIG. 7). The control antibody SP34-CEA bound to the antigen as expected (upper left). Digestion of the protein did not affect the binding (lower left).

In contrast to the control protein, all three M-TCEs demonstrated reduced binding capability to CD3δε before MMP digestion (upper panel). Digestion of the three M-TCEs by MMP9 (lower panel) partially (for the double masked M-TCE SP34-2XL4-CEA) or largely restored the binding (for the single masked M-TCEs SP34-L4-CEA and SP34-LC-L4-CEA). This result clearly demonstrated that the mask can be put on either the heavy chain, the light chain or even on both chains of SP34, albert the masking effect in the double masked M-TCE is not as strong as the two single masked M-TCE and the removal of the masks for the double masked M-TCE is not as efficacious as the single masked M-TCE.

In summary, our study led to the identification of mutants of in the CD3ε N-terminal peptide and uses of such mutant peptides to block the binding of an anti-CD3 antibody SP34. These masks are potentially useful in generating CD3-based T cell engagers by connecting them to SP34 through a protease-sensitive linker that can be activated only in an environment where the protease is abundant.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A protein construct comprising:
a. a masking peptide comprising an amino acid sequence that is at least 80% identical to the sequence of SEQ ID NO: 3 or a portion thereof, wherein the masking peptide comprises one or more amino substitutions; and
b. an antigen-binding domain,
wherein the masking peptide and the antigen-binding domain are linked via a linker.

2. The protein construct of claim 1, wherein the masking peptide comprises or consists of at least 3 amino acids (e.g., from N-terminal of CD3 [symbol] ).

3. The protein construct of claim 1 or 2, wherein the masking peptide comprises or consists of any one of SEQ ID NOs: 60-68, and wherein the masking peptide comprises an amino substitution at a position corresponding to amino acid 1, 2, or 3 of SEQ ID NO: 3.

4. The protein construct of any one of claims 1-3, wherein the amino acid substitution is one of the following:
(1) a substitution of Q at the position corresponding to amino acid 1 of SEQ ID NO: 3 with C;
(2) a substitution of D at the position corresponding to amino acid 2 of SEQ ID NO: 3 with E, G, N, or C; and
(3) a substitution of G at the position corresponding to amino acid 3 of SEQ ID NO: 3 with A or R.

5. The protein construct of claim 4, wherein the amino acid substitution is one of the following:
(1) a substitution of D at the position corresponding to amino acid 2 of SEQ ID NO: 3 with E, G, or N; and
(2) a substitution of G at the position corresponding to amino acid 3 of SEQ ID NO: 3 with A or R.

6. The protein construct of claim 4, wherein the amino acid substitution is one of the following:
(1) a substitution of D at the position corresponding to amino acid 2 of SEQ ID NO: 3 with E, or G; and
(2) a substitution of G at the position corresponding to amino acid 3 of SEQ ID NO: 3 with A.

7. The protein construct of any one of claims 1-6, wherein the masking peptide comprises or consists of any one of SEQ ID NOs: 60-68 with at most one amino acid substitution at positions corresponding to amino acid 1-3 of SEQ ID NO: 3.

8. The protein construct of claim 3, wherein the masking peptide comprises or consists of any one of SEQ ID NOs: 74, 76, 78, and 80-83.

9. The protein construct of any one of claims 1-8, wherein the linker comprises about 4 to about 18 amino acids.

10. The protein construct of any one of claims 1-9, wherein the linker comprises a protease cleavable sequence.

11. The protein construct of claim 10, wherein the protease cleavable sequence comprises an amino acid sequence of PLGL (SEQ ID NO: 85).

12. The protein construct of claim 11, wherein the linker comprises an amino acid sequence of X1-PLGL-X2, wherein
X1 comprises 0-8 glycine (G), and/or 0-8 serine (S);
X2 comprises 0-8 glycine (G), and/or 0-8 serine (S).

13. The protein construct any one of claims 1-12, wherein the linker comprises or consists of any one of SEQ ID NOs: 84-91 (e.g., any one of SEQ ID NO: 86-91).

14. The protein construct of any one of claims 1-13, wherein the masking peptide and the linker together comprise an amino acid sequence that is at least 80% identical to any one of SEQ ID NOs: 24, 26-30, 32-36, and 38-45.

15. The protein construct any one of claims 1-14, wherein the antigen-binding domain comprises a VH and a VL.

16. The protein construct of claim 15, wherein the masking peptide is linked to the VH (e.g., N-terminal of the VH) through the linker.

17. The protein construct of claim 15, wherein the masking peptide is linked to the VL (e.g., N-terminal of the VL) through the linker.

18. The protein construct of claim 15, wherein the protein construct comprises two masking peptides, wherein one of the two masking peptides is linked to the VH through a first linker, and the other of the two masking peptides is linked to the VL through a second linker.

19. The protein construct any one of claims 1-18, wherein the antigen-binding domain comprises a scFv.

20. The protein construct any one of claims 1-18, wherein the antigen-binding domain comprises a Fab.

21. The protein construct of claim 19, wherein the masking peptide is linked to the scFv (e.g., N-terminal of the scFv) through the linker.

22. The protein construct of any one of claims 1-18, wherein the antigen-binding domain comprises a VHH.

23. The protein construct of claim 22, wherein the masking peptide is linked to the VHH (e.g., N-terminal of VHH) through the linker.

24. The protein construct of any one of claims 1-23, wherein the antigen-binding domain specifically binds to CD3.

25. The protein construct of any one of claims 1-24, wherein the antigen-binding domain specifically binds to one or more epitopes in any one of SEQ ID NOs: 60-68.

26. The protein construct of any one of claims 1-25, wherein the protein construct further comprises an antigen-binding domain that specifically binds to a tumor associated antigen.

27. The protein construct of claim 26, wherein the tumor-associated antigen is carcinoembryonic antigen cell adhesion molecule 5 (CEACAM5).

28. A protein construct comprising:
(1) a first moiety comprising a masking peptide comprising an amino acid sequence that is at least 80% identical to the sequence of SEQ ID NO: 3 or a portion thereof, wherein the masking peptide comprises one or more amino substitutions;
(2) a second moiety that specifically binds to CD3; and
(3) a third moiety that specifically binds to a tumor associated antigen, wherein the first moiety and the second moiety are linked via a linker.

29. The protein construct of claim 28, wherein the masking peptide comprises or consists of at least 3 amino acids (e.g., from N-terminal of CD3 [symbol] ).

30. The protein construct of claim 28 or 29, wherein the masking peptide comprises or consists of any one of SEQ ID NOs: 60-68, and wherein the masking peptide comprises an amino substitution at a position corresponding to amino acid 1, 2, or 3 of SEQ ID NO: 3.

31. The protein construct of any one of claims 28-30, wherein the amino acid substitution is one of the following:
(1) a substitution of Q at the position corresponding to amino acid 1 of SEQ ID NO: 3 with C;
(2) a substitution of D at the position corresponding to amino acid 2 of SEQ ID NO: 3 with N, E, G, or C; and
(3) a substitution of G at the position corresponding to amino acid 3 of SEQ ID NO: 3 with A or R.

32. The protein construct of claim 31, wherein the amino acid substitution is one of the following:
(1) a substitution of D at the position corresponding to amino acid 2 of SEQ ID NO: 3 with E, G, or N; and
(2) a substitution of G at the position corresponding to amino acid 3 of SEQ ID NO: 3 with A or R.

33. The protein construct of claim 31, wherein the amino acid substitution is one of the following:
(1) a substitution of D at the position corresponding to amino acid 2 of SEQ ID NO: 3 with E, or G; and
(2) a substitution of G at the position corresponding to amino acid 3 of SEQ ID NO: 3 with A.

34. The protein construct of any one of claims 28-33, wherein the masking peptide comprises or consists of any one of SEQ ID NO: 74, 76, 78, and 80-83.

35. The protein construct of any one of claims 28-34, wherein the linker comprises a protease cleavable sequence.

36. The protein construct of claim 35, wherein the protease cleavable sequence comprises an amino acid sequence of PLGL (SEQ ID NO: 85).

37. The protein construct of claim 36, wherein the linker comprises an amino acid sequence of X1-PLGL-X2, wherein
X1 comprises 0-8 glycine (G), and/or 0-8 serine (S);
X2 comprises 0-8 glycine (G), and/or 0-8 serine (S).

38. The protein construct any one of claims 28-37, wherein the linker comprises or consists of any one of SEQ ID NOs: 84-91 (e.g., any one of SEQ ID NO: 86-91).

39. The protein construct of any one of claims 28-38, wherein the second moiety comprises an antibody or antigen-binding fragment thereof.

40. The protein construct of any one of claims 28-39, wherein the second moiety comprises an antigen-binding fragment of an antibody.

41. The protein construct of any one of claims 28-39, wherein the second moiety comprises a VHH.

42. The protein construct of any one of claims 28-41, wherein the third moiety comprises an antibody or antigen-binding fragment thereof.

43. The protein construct of any one of claims 28-42, wherein the third moiety comprises an antigen-binding fragment of an antibody.

44. The protein construct of any one of claims 28-42, wherein the third moiety comprises a VHH.

45. The protein construct of any one of claims 28-44, wherein the tumor associated antigen is selected from the group consisting of CD2, CD4, CD19, CD20, CD22, CD23, CD30, CD33, CD37, CD40, CD44v6, CD52, CD56, CD70, CD74, CD79a, CD80, CD98, CD138, EGFR (Epidermal growth factor receptor), VEGF (Vascular endothelial growth factor), VEGFR1 (Vascular endothelial growth factor receptor 1), PDGFR (Platelet-derived growth factor receptor), RANKL (Receptor activator of nuclear factor kappa-B ligand), GPNMB (Transmembrane glycoprotein Neuromedin B), EphA2 (Ephrin type-A receptor 2), PSMA (Prostate-specific membrane antigen), Cripto (Cryptic family protein 1B), EpCAM (Epithelial cell adhesion molecule), CTLA4 (Cytotoxic T-Lymphocyte Antigen 4), IGF-IR (Type 1 insulin-like growth factor receptor), GP3 (M13 bacteriophage), GP9 (Glycoprotein IX), CD42a, GP 40 (Glycoprotein 40 kDa), GPC3 (glypican-3), GPC1 (glypican-1), TRAILR1 (Tumor necrosis factor-related apoptosis-inducing ligand receptor 1), TRAILRII (Tumor necrosis factor-related apoptosis-inducing ligand receptor II), FAS (Type II transmembrane protein), PS (phosphatidyl serine) lipid, Muc (Mucin 1, PEM), Muc18, CD146, a501 integrin, α4β1 integrin, αv integrin (Vitronectin Receptor), Chondrolectin, CAIX (Carbonic anhydrase IX), GD2 gangloside, GD3 gangloside, GM1 gangloside, Lewis Y antigen, Mesothelin, HER2 (Human Epidermal Growth factor 2), HER3, HER4, FN14 (Fibroblast Growth Factor Inducible 14), CS1 (Cell surface glycoprotein, CD2 subset 1, CRACC, SLAMF7, CD319), 41BB CD137, SIP (Siah-1 Interacting Protein), CTGF (Connective tissue growth factor), HLADR (MHC class II cell surface receptor), PD-1 (Programmed Death 1, Type I membrane protein, PD-L1 (Programmed Death Ligand 1), PD-L2 (Programmed Death Ligand 2), IL-2 (Interleukin-2), IL-8 (Interleukin-8), IL-13 (Interleukin-13), PIGF (Phosphatidylinositol-glycan biosynthesis class F protein), NRP1 (Neuropilin-1), ICAM1, CD54, GC182 (Claudin 18.2), Claudin, HGF (Hepatocyte growth factor), CEA (Carcinoembryonic antigen), LTβR (lymphotoxin β receptor), Kappa Myeloma, Folate Receptor alpha, GRP78 (BIP, 78 kDa Glucose-regulated protein), A33 antigen, PSA (Prostate-specific antigen), CA 125 (Cancer antigen 125 or carbohydrate antigen 125), CA19.9, CA15.3, CA242, leptin, prolactin, osteopontin, IGF-II (Insulin-like growth factor 2), fascin, sPIgR (secreted chain of polymorphic immunoglobulin receptor), 14-3-3 eta protein, 5T4, ETA (epithelial tumor antigen), MAGE (Melanoma-associated antigen), MAPG (Melanoma-associated proteoglycan, NG2), vimentin, EPCA-1 (Early prostate cancer antigen-2), TAG-72 (Tumor-associated glycoprotein 72), factor VIII, Neprilysin (Membrane metallo-endopeptidase), 17-1 A (Epithelial cell surface antigen 17-1A), nucleolin, nucleophosmin, and any combination thereof.

46. The protein construct of claim 45, wherein the tumor associated antigen is carcinoembryonic antigen cell adhesion molecule 5 (CEACAM5).

47. The protein construct of any one of claims 28-46, wherein the second moiety is linked to a first CH2 domain and a first CH3 domain.

48. The protein construct of claim 47, wherein the second moiety is linked to the first CH2 domain and the first CH3 domain through a IgG hinge region.

49. The protein construct of any one of claims 28-48, wherein the third moiety is linked to a second CH2 domain and a second CH3 domain.

50. The protein construct of claim 49, wherein the third moiety is linked to the second CH2 domain and the second CH3 domain through a IgG hinge region.

51. The protein construct of claim 49 or 50, wherein the first and the second CH3 domains have one or more knobs-into-holes mutations.

52. A polynucleotide encoding the protein construct of any one of claims 1-51.

53. A vector comprising the polynucleotide of claim 52.

54. A cell comprising the vector of claim 53.

55. A method of producing a protein construct, the method comprising
(a) culturing the cell of claim 54 under conditions sufficient for the cell to produce the protein construct; and
(b) collecting the protein construct produced by the cell.

56. A method of inducing or activating a T cell immunity in a subject, comprising administering an effective amount of the protein construct of any one of claims 1-51 to the subject.

57. The method of claim 56, wherein the T cell immunity is induced or activated upon the cleavage of the protease cleavable domain of the linker.

58. The method of claim 57, wherein the linker is cleaved at a site of abundant protease activity.

59. The method of claim 58, wherein the site of abundant protease activity is a tumor microenvironment.

60. The method of any one of claims 57-59, wherein the protease is MMP9.

61. A method of inhibiting tumor growth in a subject, comprising administering an effective amount of the protein construct of any one of claims 1-51 to the subject.

62. A method of treating a cancer in a subject, comprising administering an effective amount of the protein construct of any one of claims 1-51 to the subject.

63. The method of any one of claims 56-62, further comprising administering an additional therapeutic agent to the subject.

64. A masking peptide comprising:
X₁X₂X₃NE (SEQ ID NO: 92) or X₁X₂X₃NEE (SEQ ID NO: 93),
wherein
X₁ is Q, or C;
X₂ is D, E, G, N, or C; and
X₃ is G, A or R.

65. The masking peptide of claim 64, wherein
X₁ is Q;
X₂ is D, E, G, or N; and
X₃ is G, A or R.

66. The masking peptide of claim 64, wherein
X₁ is Q;
X₂ is D, E, or G; and
X₃ is G, or A.

67. The masking peptide of any one of claims 64-66, wherein X₁X₂X₃NE (SEQ ID NO: 92) or X₁X₂X₃NEE (SEQ ID NO: 93) in the masking peptide is not identical to QDGNE (SEQ ID NO: 60) or QDGNEE (SEQ ID NO: 68).

68. The masking peptide of any one of claims 64-67, wherein X₁X₂X₃ in the masking peptide differs from QDG by one amino acid.


SEQUENCE LISTING (93 sequences)

SEQ ID NO: 1 - protein, 125 aa, synthetic construct
  REGION 1..125: note = VH of anti-CD3 antibody SP34
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 2 - protein, 109 aa, synthetic construct
  REGION 1..109: note = VL of anti-CD3 antibody SP34
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIK

SEQ ID NO: 3 - protein, 97 aa, synthetic construct
  REGION 1..97: note = CD3e
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARV

SEQ ID NO: 4 - protein, 10 aa, synthetic construct
  REGION 1..10: note = CD3e1-5
QDGNEGGGGS

SEQ ID NO: 5 - protein, 13 aa, synthetic construct
  REGION 1..13: note = CD3e1-8
QDGNEEMGGGGGS

SEQ ID NO: 6 - protein, 16 aa, synthetic construct
  REGION 1..16: note = CD3e1-11
QDGNEEMGGITGGGGS

SEQ ID NO: 7 - protein, 19 aa, synthetic construct
  REGION 1..19: note = CD3e1-14
QDGNEEMGGITQTPGGGGS

SEQ ID NO: 8 - protein, 22 aa, synthetic construct
  REGION 1..22: note = CD3e1-17
QDGNEEMGGITQTPYKVGGGGS

SEQ ID NO: 9 - protein, 25 aa, synthetic construct
  REGION 1..25: note = CD3e1-20
QDGNEEMGGITQTPYKVSISGGGGS

SEQ ID NO: 10 - protein, 28 aa, synthetic construct
  REGION 1..28: note = CD3e1-23
QDGNEEMGGITQTPYKVSISGTTGGGGS

SEQ ID NO: 11 - protein, 32 aa, synthetic construct
  REGION 1..32: note = CD3e1-27
QDGNEEMGGITQTPYKVSISGTTVILTGGGGS

SEQ ID NO: 12 - protein, 10 aa, synthetic construct
  REGION 1..10: note = L0-WT mask+linker
QDGNEEPLGL

SEQ ID NO: 13 - protein, 14 aa, synthetic construct
  REGION 1..14: note = L2-WT mask+linker
QDGNEEGSPLGLGS

SEQ ID NO: 14 - protein, 16 aa, synthetic construct
  REGION 1..16: note = L3-WT mask+linker
QDGNEESGSPLGLSGG

SEQ ID NO: 15 - protein, 18 aa, synthetic construct
  REGION 1..18: note = L4-WT mask+linker
QDGNEEGSGSPLGLGSGS

SEQ ID NO: 16 - protein, 20 aa, synthetic construct
  REGION 1..20: note = L5-WT mask+linker
QDGNEESSGGSPLGLSSGGS

SEQ ID NO: 17 - protein, 22 aa, synthetic construct
  REGION 1..22: note = L6-WT mask+linker
QDGNEESSGGGSPLGLSSGGGS

SEQ ID NO: 18 - protein, 24 aa, synthetic construct
  REGION 1..24: note = L7-WT mask+linker
QDGNEESSGGSGSPLGLSSGGSGS

SEQ ID NO: 19 - protein, 14 aa, synthetic construct
  REGION 1..14: note = L2-Q1E mask+linker
EDGNEEGSPLGLGS

SEQ ID NO: 20 - protein, 14 aa, synthetic construct
  REGION 1..14: note = L2-Q1S mask+linker
SDGNEEGSPLGLGS

SEQ ID NO: 21 - protein, 14 aa, synthetic construct
  REGION 1..14: note = L2-Q1Y mask+linker
YDGNEEGSPLGLGS

SEQ ID NO: 22 - protein, 14 aa, synthetic construct
  REGION 1..14: note = L2-Q1H mask+linker
HDGNEEGSPLGLGS

SEQ ID NO: 23 - protein, 14 aa, synthetic construct
  REGION 1..14: note = L2-Q1V mask+linker
VDGNEEGSPLGLGS

SEQ ID NO: 24 - protein, 14 aa, synthetic construct
  REGION 1..14: note = L2-D2N mask+linker
QNGNEEGSPLGLGS

SEQ ID NO: 25 - protein, 14 aa, synthetic construct
  REGION 1..14: note = L2-D2R mask+linker
QRGNEEGSPLGLGS

SEQ ID NO: 26 - protein, 14 aa, synthetic construct
  REGION 1..14: note = L2-D2E mask+linker
QEGNEEGSPLGLGS

SEQ ID NO: 27 - protein, 18 aa, synthetic construct
  REGION 1..18: note = L4-D2E mask+linker
QEGNEEGSGSPLGLGSGS

SEQ ID NO: 28 - protein, 20 aa, synthetic construct
  REGION 1..20: note = L5-D2E mask+linker
QEGNEESSGGSPLGLSSGGS

SEQ ID NO: 29 - protein, 22 aa, synthetic construct
  REGION 1..22: note = L6-D2E mask+linker
QEGNEESSGGGSPLGLSSGGGS

SEQ ID NO: 30 - protein, 24 aa, synthetic construct
  REGION 1..24: note = L7-D2E mask+linker
QEGNEESSGGSGSPLGLSSGGSGS

SEQ ID NO: 31 - protein, 14 aa, synthetic construct
  REGION 1..14: note = L2-D2I mask+linker
QIGNEEGSPLGLGS

SEQ ID NO: 32 - protein, 14 aa, synthetic construct
  REGION 1..14: note = L2-D2G mask+linker
QGGNEEGSPLGLGS

SEQ ID NO: 33 - protein, 18 aa, synthetic construct
  REGION 1..18: note = L4-D2G mask+linker
QGGNEEGSGSPLGLGSGS

SEQ ID NO: 34 - protein, 20 aa, synthetic construct
  REGION 1..20: note = L5-D2G mask+linker
QGGNEESSGGSPLGLSSGGS

SEQ ID NO: 35 - protein, 22 aa, synthetic construct
  REGION 1..22: note = L6-D2G mask+linker
QGGNEESSGGGSPLGLSSGGGS

SEQ ID NO: 36 - protein, 24 aa, synthetic construct
  REGION 1..24: note = L7-D2G mask+linker
QGGNEESSGGSGSPLGLSSGGSGS

SEQ ID NO: 37 - protein, 14 aa, synthetic construct
  REGION 1..14: note = L2-G3I mask+linker
QDINEEGSPLGLGS

SEQ ID NO: 38 - protein, 14 aa, synthetic construct
  REGION 1..14: note = L2-G3A mask+linker
QDANEEGSPLGLGS

SEQ ID NO: 39 - protein, 18 aa, synthetic construct
  REGION 1..18: note = L4-G3A mask+linker
QDANEEGSGSPLGLGSGS

SEQ ID NO: 40 - protein, 20 aa, synthetic construct
  REGION 1..20: note = L5-G3A mask+linker
QDANEESSGGSPLGLSSGGS

SEQ ID NO: 41 - protein, 22 aa, synthetic construct
  REGION 1..22: note = L6-G3A mask+linker
QDANEESSGGGSPLGLSSGGGS

SEQ ID NO: 42 - protein, 24 aa, synthetic construct
  REGION 1..24: note = L7-G3A mask+linker
QDANEESSGGSGSPLGLSSGGSGS

SEQ ID NO: 43 - protein, 14 aa, synthetic construct
  REGION 1..14: note = L2-G3R mask+linker
QDRNEEGSPLGLGS

SEQ ID NO: 44 - protein, 18 aa, synthetic construct
  REGION 1..18: note = L4-G3R mask+linker
QDRNEEGSGSPLGLGSGS

SEQ ID NO: 45 - protein, 22 aa, synthetic construct
  REGION 1..22: note = L6-G3R mask+linker
QDRNEESSGGGSPLGLSSGGGS

SEQ ID NO: 46 - protein, 8 aa, synthetic construct
  REGION 1..8: note = SP34 VHCDR1
GFTFNTYA

SEQ ID NO: 47 - protein, 10 aa, synthetic construct
  REGION 1..10: note = SP34 VHCDR2
IRSKYNNYAT

SEQ ID NO: 48 - protein, 16 aa, synthetic construct
  REGION 1..16: note = SP34 VHCDR3
VRHGNFGNSYVSWFAY

SEQ ID NO: 49 - protein, 9 aa, synthetic construct
  REGION 1..9: note = SP34 VLCDR1
TGAVTTSNY

SEQ ID NO: 50 - skipped

SEQ ID NO: 51 - protein, 9 aa, synthetic construct
  REGION 1..9: note = SP34 VLCDR3
ALWYSNLWV

SEQ ID NO: 52 - protein, 125 aa, synthetic construct; identical to SEQ ID NO: 1
  REGION 1..125: note = CEA VHH

SEQ ID NO: 53 - protein, 8 aa, synthetic construct
  REGION 1..8: note = CEA VHH CDR1
GFTFNTYA

SEQ ID NO: 54 - protein, 10 aa, synthetic construct
  REGION 1..10: note = CEA VHH CDR3
IRSKYNNYAT

SEQ ID NO: 55 - protein, 16 aa, synthetic construct
  REGION 1..16: note = CEA VHH CDR3
VRHGNFGNSYVSWFAY

SEQ ID NO: 56 - protein, 120 aa, synthetic construct
  REGION 1..120: note = CEA
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 57 - protein, 450 aa, synthetic construct
  REGION 1..450: note = SP34-knob
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 58 - protein, 216 aa, synthetic construct
  REGION 1..216: note = SP34-LC
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 347 aa, synthetic construct
  REGION 1..347: note = CEA-Hole
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSL

SEQ ID NO: 60 - protein, 5 aa, synthetic construct
  REGION 1..5: note = mask only
QDGNE

SEQ ID NO: 61 - protein, 8 aa, synthetic construct
  REGION 1..8: note = mask only
QDGNEEMG

SEQ ID NO: 62 - protein, 11 aa, synthetic construct
  REGION 1..11: note = mask only
QDGNEEMGGIT

SEQ ID NO: 63 - protein, 14 aa, synthetic construct
  REGION 1..14: note = mask only
QDGNEEMGGITQTP

SEQ ID NO: 64 - protein, 17 aa, synthetic construct
  REGION 1..17: note = mask only
QDGNEEMGGITQTPYKV

SEQ ID NO: 65 - protein, 20 aa, synthetic construct
  REGION 1..20: note = mask only
QDGNEEMGGITQTPYKVSIS

SEQ ID NO: 66 - protein, 23 aa, synthetic construct
  REGION 1..23: note = mask only
QDGNEEMGGITQTPYKVSISGTT

SEQ ID NO: 67 - protein, 27 aa, synthetic construct
  REGION 1..27: note = mask only
QDGNEEMGGITQTPYKVSISGTTVILT

SEQ ID NO: 68 - protein, 6 aa, synthetic construct
  REGION 1..6: note = L0-WT mask
QDGNEE

SEQ ID NO: 69 - protein, 6 aa, synthetic construct
  REGION 1..6: note = L2-Q1E mask
EDGNEE

SEQ ID NO: 70 - protein, 6 aa, synthetic construct
  REGION 1..6: note = L2-Q1S mask
SDGNEE

SEQ ID NO: 71 - protein, 6 aa, synthetic construct
  REGION 1..6: note = L2-Q1Y mask
YDGNEE

SEQ ID NO: 72 - protein, 6 aa, synthetic construct
  REGION 1..6: note = L2-Q1H mask
HDGNEE

SEQ ID NO: 73 - protein, 6 aa, synthetic construct
  REGION 1..6: note = L2-Q1V mask
VDGNEE

SEQ ID NO: 74 - protein, 6 aa, synthetic construct
  REGION 1..6: note = L2-D2N mask
QNGNEE

SEQ ID NO: 75 - protein, 6 aa, synthetic construct
  REGION 1..6: note = L2-D2R mask
QRGNEE

SEQ ID NO: 76 - protein, 6 aa, synthetic construct
  REGION 1..6: note = L2-D2E mask
QEGNEE

SEQ ID NO: 77 - protein, 6 aa, synthetic construct
  REGION 1..6: note = L2-D2I mask
QIGNEE

SEQ ID NO: 78 - protein, 6 aa, synthetic construct
  REGION 1..6: note = L2-D2G mask
QGGNEE

SEQ ID NO: 79 - protein, 6 aa, synthetic construct
  REGION 1..6: note = L2-G3I mask
QDINEE

SEQ ID NO: 80 - protein, 6 aa, synthetic construct
  REGION 1..6: note = L2-G3A mask
QDANEE

SEQ ID NO: 81 - protein, 6 aa, synthetic construct
  REGION 1..6: note = L2-Q1C mask
CDGNEE

SEQ ID NO: 82 - protein, 6 aa, synthetic construct
  REGION 1..6: note = L2-Q1C mask
QCGNEE

SEQ ID NO: 83 - protein, 6 aa, synthetic construct
  REGION 1..6: note = L2-G3R mask
QDRNEE

SEQ ID NO: 84 - protein, 5 aa, synthetic construct
  REGION 1..5: note = GGGGS linker
GGGGS

SEQ ID NO: 85 - protein, 4 aa, synthetic construct
  REGION 1..4: note = PLGL linker
PLGL

SEQ ID NO: 86 - protein, 8 aa, synthetic construct
  REGION 1..8: note = linker only
GSPLGLGS

SEQ ID NO: 87 - protein, 10 aa, synthetic construct
  REGION 1..10: note = linker only
SGSPLGLSGG

SEQ ID NO: 88 - protein, 12 aa, synthetic construct
  REGION 1..12: note = linker only
GSGSPLGLGSGS

SEQ ID NO: 89 - protein, 14 aa, synthetic construct
  REGION 1..14: note = linker only
SSGGSPLGLSSGGS

SEQ ID NO: 90 - protein, 16 aa, synthetic construct
  REGION 1..16: note = linker only
SSGGGSPLGLSSGGGS

SEQ ID NO: 91 - protein, 18 aa, synthetic construct
  REGION 1..18: note = linker only
SSGGSGSPLGLSSGGSGS

SEQ ID NO: 92-93 - skipped (2 entries)